(19) United States
(12) Patent Application Publication
(10) Pub. No.: US 2006/0183120 A1
(43) Pub. Date: Aug. 17, 2006

(54) Molecular sub-classification of kidney tumors and the discovery of new diagnostic markers

(76) Inventors: Bin Tean Teh, Ada, MI (US); Masayuki Takahashi, Tokushima (JP)

Correspondence Address: MCKENNA LONG & ALDRIDGE LLP, 1900 K STREET, NW, WASHINGTON, DC, 20006, US

(21) Appl. No.: 10/530,187

(22) PCT Filed: Oct. 6, 2003
(86) PCT No.: PCT/US03/31476

§ 371 Date: Feb. 27, 2006

(30) Foreign Application Priority Data
Oct. 4, 2002 (US) 60415775

(51) Int. Cl. C12Q 1/68; G01N 33/574; C07H 21/04; C12M 1/34; C07K 14/82

(52) U.S. Cl. 435/6; 435/7.23; 435/287.2; 530/350; 536/23.5

(57) ABSTRACT

Genes that are differentially expressed in subtypes of renal cell carcinomas are disclosed as are their polypeptide products. This information is utilized to produce nucleic acid and antibody probes and sets of such probes that are specific for these genes and their products. Methods employing these probes, including hybridization and immunological methods, are used to determine the subtype of a renal cell tumor sample from a subject based on the differential expression of such genes that is characteristic of the cancer subtype.

This application claims the benefit of the filing date of U.S. Provisional application Ser. No. 60/415,775, filed Oct. 4, 2002, which is incorporated by reference herein in its entirety.

### BACKGROUND OF THE INVENTION

1. Field of the Invention

The present invention in the field of molecular biology and medicine relates, e.g., to gene expression profiling of certain types of kidney cancer and the use of the profiles to, e.g., identify diagnostic markers in patients.

2. Description of the Background Art

Renal cell carcinoma (RCC) is the most common malignancy of the adult kidney, representing 2% of all malignancies and 2% of cancer-related deaths. The incidence of RCC is increasing and the increase cannot be explained by the increased use of abdominal imaging procedures alone. (Chow et al., JAMA 1999; 281(17): 1628-31).

RCC is a clinicopathologically heterogeneous disease, traditionally subdivided into clear cell, granular cell, papillary, chromophobe, spindle cell, cystic, and collecting duct carcinoma, based on morphological features according to the WHO International Histological Classification of Kidney Tumors (Mostfi, F K et al., 1998). Clear cell RCC(CC-RCC) is the most common adult renal neoplasm, representing 70% of all renal neoplasms, and is thought to originate in the proximal tubules. Papillary RCC accounts for 10-15%, chromophobe RCC 4-6%, collecting duct carcinoma<1%, and unclassified 4-5% of RCC. Spindle RCC, also called sarcomatoid RCC, is characterized by prominent spindle cell features, and is thought to represent the high-grade end of the subgroups. Granular cell RCC, which is no longer considered a subtype in the current classification systems, is still being used by many pathologists around the world. Instead, granular RCC can often be reclassified into other subtypes (Storkel et al., Cancer 1997; 80: 987-9).

With recent advances in molecular genetics, the subtypes of RCC have been associated with distinct genetic abnormalities. This association has led to a proposal for molecular diagnosis of RCC (Bugert et al., Am J Pathol 1996; 149:2081-2088). The majority of clear cell RCC, for example, has a loss of chromosome 3 and inactivating mutations of the VHL gene, whereas papillary RCC are frequently associated with trisomy of chromosomes 3q, 7, 12, 16, 17 and 20, and loss of the Y chromosome. A portion of them also harbor MET mutations. It has been proposed that, even in the absence of prominent papillae, these aberrant chromosomal features could support the diagnosis of papillary RCC. Conversely, kidney cancers that do not possess these genetic characteristics should not be designated as papillary RCC even when papillary structures are prominent (Storkel et al., 1997 supra). Frequent loss of sex chromosomes, chromosomes 1 and 14 have been found in renal oncocytoma, a rarely metastasizing entity composed of acinar-arranged, large eosinophilic cells (Presti et al., Genes Chromosomes Cancer 1996; 17:199-204). Accurate subtyping of renal tumors is important for predicting prognosis and designing treatment for patients.

Microarray technology can provide insights into underlying molecular mechanisms of many types of cancers. Gene expression profiles obtained with microarray technology can serve as the molecular signatures of cancer, and may be used to distinguish among histological subtypes as well as the discovery of novel distinct subtypes that correlate with clinical parameters. Such distinctions may reflect, e.g., the heterogeneity in transformation mechanisms, cell types, or aggressiveness among tumors. For example, approximately 100 genes were identified as differentially expressed in serous ovarian cancers as compared to mucinous type (Ono et al., Cancer Res 2000; 60(18):5007-11). Other studies have identified distinct gene sets that distinguish between acute myeloid leukemia and acute lymphoblastic leukemias (Golub et al., Science 1999; 286:531-537), between hereditary breast cancer with BRCA1 and BRCA2 mutations (Hedenfalk et al., N. Engl J Med 2001; 344:539-548), between hepatitis-B and hepatitis C-positive hepatocellular carcinomas (Okabe et al., Cancer Res 2001; 61:2129-37) and between diffuse large B-cell lymphoma with good and poor prognosis.

In general, diagnosis of RCC is currently performed by histologic analysis. Corporal imaging methods, e.g., ultrasonography, CT scans and X-rays, are also used. These modalities lack the rigor to distinguish fully among the various types of RCCs, and are sometimes slow and laborious. The marked heterogeneity of RCCs provides a great challenge in diagnosis and treatment. This complicates prognosis and hinders selection of the most appropriate therapy. There is a need for additional methods that can supplement or supplant the available diagnostic approaches for differentiating among the types of RCC.

### DESCRIPTION OF THE INVENTION

The present invention relates, e.g., to the identification of genes and gene products (molecular markers) whose expression is upregulated in a large percentage of RCCs of a particular sub-type, e.g., CC-RCC, papillary RCC, chromophobe-RCC/oncocytoma, sarcomatoid-RCC, TCC, or Wilms' tumor (WT), compared to a baseline value. As used herein, a “baseline value” includes, e.g., the expression in other types of RCC or normal renal tissue, such as from the same subject or from a “pool” of normal subjects, whether obtained at the same time as a sample from an RCC, or available in a generic database. For example, about 30 molecular markers are identified herein as significantly more highly expressed in CC-RCC than in the other subtypes studied or in normal kidney tissue; about 30 such molecular markers are identified for papillary-RCC; about 30 such molecular markers are identified for chromophobe-RCC/oncocytoma-RCC; about 29 such molecular markers are identified for sarcomatoid-RCC; about 74 such molecular markers are identified for TCC; and about two such molecular markers are identified for Wilms' tumor.

These molecular markers (molecular signatures) can serve as the basis for diagnostic assays to distinguish among these sub-types of RCCs. For example, nucleic acid probes corresponding to one or more of the overexpressed genes, and/or antibodies specific for proteins encoded by them, can be used to analyze a sample from a renal tumor, in order to determine to which subtype the tumor belongs. Assays of this type can detect the differential expression of certain selected genes, expressed sequence tags (ESTs), gene fragments, mRNAs, and other polynucleotides as described herein. In a preferred embodiment, the samples are tissues (e.g., sections of paraffin-embedded blocks) or tissue extracts (e.g., preparations of nucleic acid and/or protein). The overexpressed genes and gene products can also serve to identify therapeutic targets, e.g. genes which are commonly overexpressed in one of the renal cancer subtypes, or proteins whose activity is enhanced. For example, one can focus on developing drugs that (1) suppress up-regulation, for example by acting on a cellular pathway that stimulates expression of this gene, (2) act directly on the protein product, or (3) bypass the step in a cellular pathway mediated by the product of this gene. The overexpressed genes can also provide a basis for explaining the different metabolic processes exhibited by the different sub-types of renal tumors, and can be used as research tools.

One aspect of the invention is a composition (combination) comprising
(a) at least about one, two, five or ten isolated nucleic acids from the set represented by SEQ ID NOs: 1-30 from Table 1, or fragments thereof which nucleic acids hybridize specifically to the nucleic acids of genes that are overexpressed (upregulated) in a large percentage of CC-RCC, and/or
(b) at least about one, two, five or ten isolated nucleic acids from the set represented by SEQ ID NOs: 31-60 from Table 2, or fragments thereof which nucleic acids hybridize specifically to the nucleic acids of genes that are overexpressed (upregulated) in a large percentage of papillary-RCC), and/or
(c) at least about at least about one, two, five or ten isolated nucleic acids from the set represented by SEQ ID NOs: 61-90 from Table 3, or fragments thereof which nucleic acids hybridize specifically to the nucleic acids of genes that are overexpressed (upregulated) in a large percentage of chromophobe RCC, and/or
(d) at least about at least about one, two, five or ten isolated nucleic acids from the set represented by SEQ ID NOs: 91-119 from Table 5, or fragments thereof. These nucleic acids hybridize specifically to the nucleic acids of genes that are overexpressed (upregulated) in a large percentage of sacomatoid RCC), and/or
(e) at least about at least about one, two, five or ten isolated nucleic acids from the set represented by SEQ ID NOs: 120-193 from Table 6, or fragments thereof. (These nucleic acids hybridize specifically to the nucleic acids of genes that are overexpressed (upregulated) in a large percentage of TCC), and/or
(f) one or two isolated nucleic acids from the set represented by SEQ ID NOs: 194 and 195, or fragments thereof which nucleic acids hybridize specifically to the nucleic acids of genes that are overexpressed (upregulated) in a large percentage of Wilms' tumor).
   In one embodiment of this invention, nucleic acid sequences corresponding to genes that have been previously reported to be differentially overexpressed in CC-RCC, papillary RCC, chromophobe-RCC/oncocytoma, sarcomatoid RCC, TCC, or Wilms' tumors are excluded from the composition described above.

The length of each of the preceding nucleic acid fragments in the above combinations is preferably at least about 8 or at least about 15 contiguous nucleotides of the sequences. As used herein, the term “preferably” is to be understood to mean “not necessarily.”

The preceding nucleic acids (represented by the SEQ ID NOs) can be used as probes to identify (e.g., by hybridization assays) polynucleotides that are overexpressed in the indicated RCC subtypes. A skilled worker will recognize how to select suitable fragments of those nucleic acids that will also hybridize specifically to the polynucleotides of interest.

As noted, combination (a), (b), (c), (d), or (e) above may comprise any combination of, e.g., about 5, 8, or 10 nucleic acids from each of the indicated sets of nucleic acids (from Tables 1, 2, 3, 5 and 6, respectively). Preferably, the nucleic acids in such a set or “subgroup” share a common core structure, a common function or another property.

More specifically, the isolated nucleic acids of a composition of the invention may comprise 1 or any combination of 2, 3, 4, or 5 nucleic acids represented by each of the following groups of sequences:
(a) SEQ ID NO:1; SEQ ID NO:2; SEQ ID NO:3; SEQ ID NO:5; and/or SEQ ID NO:6 (preferably all five nucleic acids are present); and/or
(b) SEQ ID NO:31; SEQ ID NO:33; SEQ ID NO:34; SEQ ID NO:35; and/or SEQ ID NO:36; (preferably all five nucleic acids are present); and/or
(c) SEQ ID NO:61; SEQ ID NO:62; SEQ ID NO:64; SEQ ID NO:65; and/or SEQ ID NO:66; (preferably all five nucleic acids are present); and/or
(d) SEQ ID NO:91; SEQ ID NO:92; SEQ ID NO:93; SEQ ID NO:94; and/or SEQ ID NO:95; (preferably all five nucleic acids are present); and/or
(e) SEQ ID NO:120; SEQ ID NO:121; SEQ ID NO:122; SEQ ID NO:123; and/or SEQ ID NO:125; (preferably all five nucleic acids are present), and/or
(f) one or two of SEQ ID NO:194 and/or SEQ ID NO:195,
   and/or a fragment that comprises at least about 8 or at least about 15 contiguous nucleotides of any one of the above sequences.

In one embodiment, the fifth nucleic acid in (e) is SEQ ID NO:124.

As used herein, the singular forms “a,” “an,” and “the” include plural referents unless the context clearly dictates otherwise. For example, “a” fragment, as used above, means one or more fragments, which can include, e.g., fragments of two different nucleic acids.

In another aspect, a composition of the invention may comprise a set of two or more nucleic acids (e.g., polynucleotide probes), each of which hybridizes with part or all of a coding sequence that is up-regulated (overexpressed) in CC-RCC, papillary RCC, chromophobe/oncocytoma RCC, sarcomatoid RCC, TCC, or Wilms' tumors, compared to a baseline value. The composition may comprise, e.g., a set of at least about five of these nucleic acids, or a set of at least about ten of these nucleic acids.

In the nucleic acid compositions of the invention, one or more phosphates in the helix may be modified, for example, as a phosphorothioate, a phosphoridothioate, a phosphoramidothioate, a phosphoramidate, a phosphordiimidate, a methylsphosphonate, an alkyl phosphotriester, 3′-aminopropyl, a formacetal, or an analogue thereof. The isolated nucleic acid may be of mammalian, preferably of human origin.

One embodiment of the invention is a composition comprising molecules (e.g., nucleic acids, proteins or antibodies) in the form of an array, preferably a microarray. A further discussion of arrays is presented below. A nucleic acid array may further comprise, bound to one or more nucleic acids of the array, one or more polynucleotides from a skample comprising expressed genes. The sample may be from an individual subject's renal tumor, from a normal tissue, or both. In one embodiment, the nucleic acids in an array and the polynucleotide(s) from a sample of expressed genes have been subjected to nucleic acid hybridization under high stringency conditions (such that nucleic acids of the array that are specific for particular polynucleotides from the sample are specifically hybridized to those polynucleotides).

By the term an “isolated” nucleic acid (or polypeptide, or antibody) is meant herein a nucleic acid (or polypeptide, or antibody) that is in a form other than it occurs in nature, for example in a buffer, in a dry form awaiting reconstitution, as part of an array, a kit or a pharmaceutical composition, etc. By a sequence “corresponding to” a gene, or “specific for” a gene, is meant a sequence that is substantially similar to (e.g., hybridizes under conditions of high stringency to) one of the strands of the double stranded form of that gene. By hybridizing “specifically” is meant herein that two components e.g. an expressed gene or polynucleotide and a nucleic acid. e.g., a probe, bind selectively to each other and not generally to other components to which binding is not intended. The conditions for such specific interactions can be determined routinely by one skilled in the art.

Another embodiment of the invention is a combination (composition) comprising polypeptides that are of a size and structure that can be recognized and bound by an antibody or other selective binding partner. Specifically the combination (composition) comprises:
(a) at least about one, two, five or ten isolated polypeptides each encoded by a nucleic acid from the set represented by SEQ ID NOs: 1-30 from Table 1, or antigenic fragments that comprise at least about 8 or at least about 12 contiguous amino acids of said polypeptides, and/or
(b) at least about one, two, five or ten isolated polypeptides each encoded by a nucleic acid from the set represented by SEQ ID NOs: 31-60 from Table 2, or antigenic fragments that comprise at least about 8 or at least about 12 contiguous amino acids of said polypeptides, and/or
(c) at least about one, two, five or ten isolated polypeptides each encoded by a nucleic acid from the set represented by SEQ ID NOs: 61-90 from Table 3, or antigenic fragments that comprise at least about 8 or at least about 12 contiguous amino acids of said polypeptides, and/or
(d) at least about one, two, five or ten isolated polypeptides each encoded by a nucleic acid from the set represented by SEQ ID NOs: 91-119 from Table 5, or antigenic fragments that comprise at least about 8 or at least about 12 contiguous amino acids of said polypeptides, and/or
(e) at least about one, two, five or ten isolated polypeptides each encoded by a nucleic acid from the set represented by SEQ ID NOs: 120-193 from Table 6, or antigenic fragments that comprise at least about 8 or at least about 12 contiguous nucleotides of said polypeptides, and/or
(f) one or two isolated polypeptides each encoded by a nucleic acid from the set represented by SEQ ID NOs: 194 and 195, or antigenic fragments that comprise at least about 8 or at least about 12 contiguous amino acids of said polypeptides.

Combination (a), (b), (c), (d) or (e) above may comprise any combination of, e.g., about any 5, 8, or 10 polypeptides from each of the indicated sets of polypeptides. Preferably, the polypeptides in such a subgroup share a common core structure, a common function or another property.

More specifically, the isolated polypeptides of a composition of the invention may comprise 1 or any combination of 2, 3, 4, or 5 polypeptides encoded by the nkucleic acids represented by each of the following sets of sequences:
(a) SEQ ID NO:1; SEQ ID NO:2; SEQ ID NO:3; SEQ ID NO:5; and/or SEQ ID NO:6; (preferably all five polypeptides are present); and/or
(b) SEQ ID NO:31; SEQ ID NO:33; SEQ ID NO:34; SEQ ID NO:35; and/or SEQ ID NO:36; (preferably all five polypeptides are present); and/or
(c) SEQ ID NO:61; SEQ ID NO:62; SEQ ID NO:64; SEQ ID NO:65; and/or SEQ ID NO:66; (preferably all five polypeptides are present); and/or
(d) SEQ ID NO:91; SEQ ID NO:92; SEQ ID NO:93; SEQ ID NO:94; and/or SEQ ID NO:95; (preferably all five polypeptides are present); and/or
(e) SEQ ID NO:120; SEQ ID NO:121; SEQ ID NO:122; SEQ ID NO:123; and/or SEQ ID NO:125; (preferably all five polypeptides are present); and/or
(f) one or two of SEQ ID NO:194 and/or SEQ ID NO:195;
   and/or an antigenic fragment that comprises at least about 8 or at least about 12 contiguous amino acids of the above polypeptides.
   In one embodiment, the fifth polypeptide in (e) is encoded by an ORF of SEQ ID NO:124.

A skilled worker can readily determine the amino acid sequence encoded by an open reading frame of any of the nucleic acids noted above.

For example, one embodiment of the invention is a combination (composition) comprising the following polypeptides:
(a) at least about one, two, five or ten isolated polypeptides from the set represented by SEQ ID NOs: 196-220 from Table 1, or antigenic fragments thereof that comprise at least about 8 or at least about 12 contiguous amino acids of said polypeptide sequences, and/or
(b) at least about one, two, five or ten isolated polypeptides from the set represented by SEQ ID NOs: 221-247 from Table 2, or antigenic fragments thereof that comprise at least about 8 or at least about 12 contiguous amino acids of said polypeptide sequences, and/or
(c) at least about one, two, five or ten isolated polypeptides from the set represented by SEQ ID NOs: 248-270 from Table 3, or antigenic fragments thereof that comprise at least about 8 or at least about 12 contiguous amino acids of said sequences, and/or
(d) at least about one, two, five or ten isolated polypeptides from the set represented by SEQ ID NOs: 271-296 from Table 5, or antigenic fragments thereof that comprise at least about 8 or at least about 12 contiguous amino acids of said sequence(s)

The composition may also include any of the polypeptides indicated above as being encoded by one of the mentioned nucleic acids (e.g., the polypeptides of e and f).

Each of (a), (b), (c), (d) or (e) above may comprise any combination of, (e.g., about any 5, 8, or 10 polypeptides from each of the indicated sets of polypeptides. Preferably (but not necessarily), the polypeptides in such a subgroup share a common core structure, or a common function or other property.

More specifically, the isolated polypeptides of a composition of the invention may comprise any combination of 1, 2, 3, 4, or 5 polypeptides represented by the following sets of sequences:
(a) SEQ ID NO:196; SEQ ID NO:197; SEQ ID NO:198; SEQ ID NO:199 or 200; and/or SEQ ID NO:201; (preferably all five polypeptides are present); and/or
(b) SEQ ID NO:221; SEQ ID NO:222; SEQ ID NO:223; SEQ ID NO:224; and/or SEQ ID NO:225; (preferably all five polypeptides are present); and/or
(c) SEQ ID NO:248; SEQ ID NO:249; SEQ BD NO:250; SEQ ID NO:251; and/or SEQ ID NO:252; (preferably all five polypeptides are present); and/or
(d) a polypeptide encoded by an ORF of SEQ ID NO:91 (ubiquitin thiolesterase); SEQ ID NO:271 or 272; SEQ ID NO:273; a polypeptide encoded by an ORF of SEQ ID NO:94 (H. sapiens α-1 (VI) collagen); and/or SEQ ID NO:274; (preferably all five polypeptides are present); and/or
(e) a polypeptide encoded by an ORF of SEQ ID NO:120 (keratin 14); or of SEQ ID NO:121 (collagen type VII, alpha1); or of SEQ ID NO:122 (keratin 19); or of SEQ ID NO:123 (plexin B3) and/or of SEQ ID NO:125 (integrin beta4); (preferably all 5 polypeptides are present) [in one embodiment, the polypeptide is encoded by an ORF of SEQ ID NO:124 (similar to rat collagen alpha1 (MD chain); and/or
(f) a polypeptide encoded by SEQ ID NO:194 (heparin sulfate proteoglycan) and/or by SEQ ID NO:195 (IGF II);
   and/or an antigenic fragment thereof. Such a fragment may comprise at least about 8 or at least about 12 contiguous amino acids of the above sequences.

Another aspect of the invention is a composition comprising an antibody or a combination of antibodies specific for the polypeptides described herein which may be used for the same purposes as the polypeptides. As used herein, an antibody that is “specific for” a polypeptide includes an antibody that binds selectively to the polypeptide and not generally to other polypeptides to which binding is not intended. The conditions for such specificity can be determined routinely using conventional methods.

One aspect of the invention is a composition comprising selected numbers of such antibodies in a form that permits their binding to the polypeptides for which they are specific. Such a composition may comprise:
(a) at least about one, two, five or ten isolated antibodies that are specific for polypeptides encoded by nucleic acids represented by SEQ ID NOs: 1-30 from Table 1, or specific for antigenic fragments thereof, and/or
(b) at least about one, two, five or ten isolated antibodies that are specific for polypeptides encoded by nucleic acids represented by SEQ ID NOs: 31-60 from Table 2, or specific for antigenic fragments thereof, and/or
(c) at least about one, two, five or ten isolated antibodies that are specific for polypeptides encoded by nucleic acids represented by SEQ ID NOs: 61-90 from Table 3, or specific for antigenic fragments thereof, and/or
(d) at least about one, two, five or ten isolated antibodies that are specific for polypeptides encoded by nucleic acids represented by SEQ ID NOs: 91-119 from Table 5, or specific for antigenic fragments thereof, and/or
(e) at least about one, two, five or ten isolated antibodies that are specific for polypeptides encoded by nucleic acids represented by SEQ ID NOs: 120-193 from Table 6, or specific for antigenic fragments thereof, and/or
(f) one or two isolated antibodies that are specific for polypeptides encoded by nucleic acids represented by SEQ ID NOs: 194-195, or specific for antigenic fragments thereof.
   Here too, the fragments preferably comprise at least about 8 or about 12 contiguous amino acid residues of the polypeptide.

The antibodies in any of the above compositions (including subsets) may be provided in the form of an array, such as a microarray.

This invention is also directed to a method for detecting (e.g., measuring, or quantitating) one or more polynucleotides, or polypeptides encoded by those polynucleotides, in a sample, such as a sample from an RCC tumor. The method comprises contacting the sample with a composition of nucleic acids, or of antibodies, of the invention, under conditions which permit (a) binding of the nucleic acids to the sample polynucleotides (such as hybridization under conditions of high stringency), or (b) binding of the antibodies to sample polypeptides. The method further comprises detecting the sample polynucleotides or antibodies which have bound. Preferably, the polynucleotides or polypeptides that are ones which are overexpressed (upregulation) in the sample and are indicative of a specific subtype of RCC. Detection of the polynucleotides or polypeptides thus identify the specific subtype of the RCC.

The invention provides a method for determining the subtype of a RCC in a subject, comprising
(a) hybridizing a nucleic acid composition of the invention, under conditions of high stringency, to a polynucleotide sample obtained from the renal carcinoma of the subject (the sample may be in the form of a tissue fragment or extract); and
(b) comparing the amount of one or more of the sample polynucleotides hybridized to one or more nucleic acids in the composition to a baseline value of hybridization.

The baseline value may be obtained, for example, by hybridizing the nucleic acid composition, under conditions of high stringency, to polynucleotides from normal kidney tissue, e.g., from the same subject or from a “pool” of normal individuals. Alternatively, the baseline value may be obtained from an existing database of such values.

The amount of a sample polynucleotide hybridized to a nucleic acid in the composition generally reflects the level of, i.e., the expression of, the polynucleotide in the renal tumor.

Another embodiment is a method for determining the subtype of an RCC in a subject, comprising:
(a) examining expression in RCC tumor tissue from the subject of polynucleotides that hybridize at high stringency conditions with at least one or at least two nucleic acids, or fragments thereof, which nucleic acids are described herein as being overexpressed or upregulated in a particular type of kidney tumor,
(b) examining expression in the subject's normal kidney tissue of polynucleotides that hybridize at high stringency conditions with the nucleic acids noted in (a); and
(c) comparing the expression in tumor tissue in (a) with the expression in normal tissue in (b).

In further embodiments of the above methods for determining the subtype of a renal cell carcinoma, the polynucleotide from tumor (and, optionally, from normal tissue) is labeled with a detectable label, such as a fluorescent label.

Other embodiments of the above methods are based on a relationship between a particular level of expression of particular DNA sequences (represented, e.g., by a particular level of hybridization) as being diagnostic of the RCC subtype. Examples of such relationships are:
(i) when expression, determined by hybridization to nucleic acids represented by SEQ ID NOs: 1-30, is up-regulated, e.g., at least about 5-fold, in tumor tissue compared to normal kidney tissue, the renal tumor is CC-RCC,
(ii) when the expression, determined by hybridization to nucleic acids represented by SEQ ID NOs: 31-60 is up-regulated, e.g., at least about 3-fold, in tumor tissue compared to normal kidney tissue, then the renal tumor is papillary RCC,
(iii) when the expression, determined by hybridization to nucleic acids polynucleotides represented by SEQ ID NOs: 61-90, is up-regulated, e.g., at least about 5-fold, in tumor tissue compared to normal kidney tissue, then the renal tumor is chromophobe-RCC/oncocytoma,
(iv) when the expression, determined by hybridization to nucleic acids represented by SEQ ID NOs: 91-119 is up-regulated in tumor tissue compared to normal kidney tissue, then the renal tumor is sarcomatoid-RCC,
(v) when the expression, determined by hybridization to nucleic acids represented by SEQ ID NOs: 120-193 is up-regulated in tumor tissue compared to normal kidney tissue, then the renal tumor is transitional cell carcinoma (TCC), and
(vi) when the expression, determined by hybridization to nucleic acids represented by SEQ ID NOs: 194-195 is up-regulated in tumor tissue compared to the normal kidney tissue, the renal tumor is Wilms' tumor (WT).

Another aspect of the invention is a method for determining the subtype of an RCC in a subject, comprising detecting one or more polypeptide (protein) products whose expression is upregulated in a majority of subjects with a subtype of RCC as discussed herein. Such detecting includes determining the presence of, and/or measuring the amount of the polypeptide.

Another aspect of the invention is a method for determining the subtype of an RCC in a subject, comprising
(a) contacting an antibody composition of the invention with a polypeptide sample obtained from a renal carcinoma under conditions effective for the at least one of the antibodies to bind specifically to a polypeptide for which it is specific; and
(b) comparing the amount of binding of the one or more of the polypeptides in the sample to the one or more antibodies in the composition to a baseline value.
   The sample may be a tissue fragment or extract.

The baseline value may be obtained, for example, by contacting the antibody composition, under similar conditions, to a polypeptide sample obtained from normal kidney tissue, e.g., from the same subject or from a “pool” of normal individuals.

The amount of sample polypeptide bound to an antibody specific for it in the antibody composition generally reflects the level of expression of the polypeptide in the renal tumor.

For example, one embodiment is a method for determining the subtype of an RCC in a subject, comprising
(a) contacting RCC tissue or an extract thereof with
   (i) an antibody specific for one polypeptide or antibodies specific for two or more polypeptides encoded by nucleic acids represented by SEQ ID NOs: 1-30 from Table 1, or antibodies specific for a fragment of the polypeptide(s), under conditions in which the antibody or antibodies bind specifically to proteins that are relatively overexpressed in CC-RCC, and/or
   (ii) an antibody specific for one polypeptide or antibodies specific for two or more polypeptides encoded by nucleic acids represented by SEQ BD NOs: 31-60 from Table 2, or antibodies specific for a fragment of the polypeptide(s), under conditions in which the antibody or antibodies bind specifically to proteins that are relatively overexpressed in papillary RCC, and/or
   (iii) an antibody specific for one polypeptide or antibodies specific for two or more polypeptides encoded by nucleic acids represented by SEQ ID NOs: 61-90 from Table 3, or antibodies specific for a fragment of the polypeptide(s), under conditions in which the antibody or antibodies bind specifically to proteins that are relatively overexpressed in chromophobe RCC/oncocytoma, and/or
   (iv) an antibody specific for one polypeptide or antibodies specific for two or more polypeptides encoded by nucleic acids represented by SEQ ID NOs: 92, 93 and/or 103 or antibodies specific for a fragment of the polypeptide(s), under conditions in which the antibody or antibodies bind specifically to proteins that at relatively overexpressed in sarcomatoid RCC, and/or
   (v) an antibody specific for one polypeptide or antibodies specific for two or more polypeptides encoded by nucleic acids represented by SEQ ID NOs: 120, 121, 122, 125 and/or 126, or antibodies specific for a fragment of the polypeptide(s), under conditions in which the antibody or antibodies bind specifically to proteins that at relatively overexpressed in TCC, and/or
   (vi) an antibody specific for one or both polypeptides encoded by nucleic acids represented by SEQ ID NOs: 194-195, or antibodies specific for a fragment of the polypeptide(s), under conditions in which the antibody or antibodies bind specifically to proteins that at relatively overexpressed in Wilms' tumor,
(b) detecting or measuring the antibodies bound to said tissue or extract;,
(c) contacting a normal kidney tissue or an extract thereof obtained, e.g., from said subject or from a pool of normal kidney tissue, with one or more of said antibodies of (a)(i)-(a)(vi),
(d) detecting or measuring the antibodies bound to said normal kidney tissue or extract, and
(e) comparing the amount of binding in (b) and (d).

In other embodiments, any of the antibody compositions described herein (e.g., a subset of the antibodies) may be substituted for the antibodies described in (a)(i)-(a)(vi) above.

In any of the above methods for determining the RCC subtype, the composition may be in the form of an array, such as a microarray.

Another aspect of the invention is a kit comprising a composition of nucleic acids of the invention (e.g., in the form of an array) and, optionally, one or more reagents that facilitate hybridization of the nucleic acid in the composition to a test polynucleotide, or that facilitate detection of the test polynucleotide (e.g., detection of fluorescence). The kit may comprise an array of nucleic acids of the invention, means for carrying out hybridization of the nucleic acid in the array to a test polynucleotide of interest, and means for reading hybridization results. Hybridization results may be units of fluorescence.

Another kit comprises a composition of antibodies of the invention (e.g., in the form of an array) and, optionally, one or more reagents that facilitate binding of the antibodies with test polypeptides, or that facilitate detection of antibody binding.

Kits of the invention may comprise instructions for carrying out the hybridization or antibody binding.

Other optional elements of the present kits include suitable buffers, culture medium components, or the like; a computer or computer-readable medium for storing and/or evaluating the assay results; containers; or packaging materials. Reagents for performing suitable controls may also be included. The reagents of the kit may be in containers in which the reagents are rendered stable, e.g., in lyophilized form or stabilized liquids. The reagents may also be in single use form, e.g., in single reaction form for diagnostic use.

As used herein, the terms “nucleic acid” and “polynucleotide” refer to both DNA (including cDNA) and RNA, as well as peptide nucleic acids (PNA) or locked nucleic acids (LNA). The terms nucleic acid and polynucleotide are not intended to be limited to a particular number of nucleotides, and therefore overlap in length with oligonucleotides. Nucleic acid for gene expression analysis include those comprising ribonucleotides, deoxyribonucleotides, both, or their analogues as described below. A probe may be or may comprise a nucleic acid, without limitation of length. Preferred lengths are described below. Nucleic acids of the invention include double stranded and partially or completely single stranded molecules. In a preferred embodiment, probes for gene expression comprise single stranded nucleic acid molecules that are complementary to an mRNA target expressed by a gene of interest, or that are complementary to the opposite strand (e.g., complementary to a first strand cDNA generated from the mRNA).

The present invention uses nucleic acids to probe for, and to determine the relative expression of, target genes (referred to more generally as polynucleotides) of interest in a tissue sample, or in an extract thereof. Preferred tissue is renal tumor tissue. Expression is compared to expression of that same target in a different type of renal tumor or in normal kidney tissue.

A composition comprising nucleic acids of the invention can take any of a variety of forms. For example, the combination of isolated nucleic acids can be in a solution (e.g., an aqueous solution), and can be subjected to hybridization in solution to polynucleotides from a sample of interest. Methods of solution hybridization are well-known in the art.

Alternatively, the nucleic acids can be in the form of an array. The term “array” as used herein means an ordered (e.g., geometrically ordered) arrangement of addressable and accessible, spatially discrete and identifiable, molecules disposed on a surface. Arrays, generally described as macroarrays or microarrays, can comprise any number of individual probe sites, from about 5 to, in the case of a “microarray,” as many as about 900 or more probes. Macroarrays contain sample spots of about 300 μm diameter or larger and can be easily imaged by existing gel and blot scanners. Sample spot sizes in microarrays are typically <200 μm in diameter, and these arrays usually contains thousands of spots. Microarrays require specialized robotics and imaging equipment that generally are commercially available and well-known in the art.

Any suitable, compatible surface can be used in conjunction with this invention. The surface usually a solid, can be made of any of a variety of organic or inorganic materials or combinations thereof, including, for example, a plastic such as polypropylene or polystyrene; a ceramic; silicon; (fused) silica, quartz or glass, which can have the thickness of, for example, a glass microscope slide or a glass cover slip; paper, such as filter paper; diazotized cellulose; nitrocellulose; nylon membrane; or polyacrylamide gel pad. Substrates that are transparent to light are useful when employed with optical detection methods. In one embodiment, the surface is the plastic surface of a multiwell e.g. tissue culture dish, such as a 9k6 (or greater)-well microplate. The shape of the surface is not critical. It can, for example, be a flat square, rectangular, or circular surface; a curved surface; or a three dimensional surface such as a bead, particle, strand, precipitate, tube, sphere; etc. Microfluidic devices are also encompassed by the invention.

In a preferred embodiment, a composition comprising nucleic acids is in the form of a microarray. Microarrays are orderly arrangements of spatially resolved samples or probes (e.g., cDNAs or oligonucleotides of known sequence, ranging in size from about 15 to about 2000 nucleotides), that allow for massively parallel gene expression analysis (Lockhart D J et al., Nature (2000) 405(6788):827-836). The probes are preferably immobilized to a solid substrate and are available to hybridize with complementary polynucleotide strands (Phimister, Nature Genetics (1999) 21(supp):1-60).

The underlying concept of array hybridization analysis depends on base-pairing (hybridization) following the rules of Watson-Crick base pairing. Microarray technology adds automation to the process of resolving nucleic acids of particular identity and sequence present in an analyte sample by labeling, preferably with fluorescent labels, and subsequent hybridization to their complements immobilized to a solid support in microarray format.

The materials for a particular application are not necessarily available in convenient in kit form. The present invention provides arrays, including microarrays, that are useful for the analysis of RCC samples and the determination of the subclass of a renal tumor.

DNA microarrays (DNA “chips”) are fabricated by high-speed robotics, preferably on glass (though nylon and other plastic substrates are used). An experiment with a single DNA chip can provide simultaneous information on thousands of genes—a dramatic increase in throughput (Reichert et al. (2000) Anal. Chem. 72:6025-6029) when compared to traditional methods.

Two DNA microarray formats are preferred.
Format I: a cDNA probe (e.g., 500-5,000 bases) is immobilized to a solid surface such as glass using robotic spotting and exposed to a set of targets either separately or in a mixture. This method is traditionally called “DNA microarray” (Ekins, R et al., Trends in Biotech (1999) 17:217-218).
Format II: an array of probes that are “natural” oligo- or polynucleotides (oligomers of 20˜80 bases), oligonucleotide analogues e.g., with phosphorothioate, methylphosphonate, phosphoramidate, or 3′-aminopropyl backbones), or peptide-nucleic acids (PNA)
   Probes may be synthesized either in situ (on-chip) or by conventional synthesis followed by on-chip immobilization.

The array is (1) exposed to an analyte comprising a detectable labeled, preferably fluorescent, sample nucleic acid (typically DNA), (2) allowed to hybridize, and (3) the identity and/or abundance of complementary sequences is determined.

| 1. Probe (cDNA or | 2. Chip | 3. Target | | |
|---|---|---|---|---|
| oligonucleotide of | fabrication (putting | (detectably labeled | | |
| known identity) | probes on the chip) | sample) | 4. Assay | 5. Readout |
| Small oligos, cDNA, | Photolithography, | PolyA-mRNA | Hybridization, long, | Fluorescence, |
| chromosome | pipette, drop-touch, | extraction, RT-PCR, | short, ligase, base | radioactivity, |
| | piezoelectric (ink- | cDNA isolation, | addition, electric, MS, | etc. |
| | j0et), electric | melting | electrophoresis, flow | |
| | | | cytometry, PCR-Direct, | |
| | | | TaqMan ®, etc. | |

One embodiment of the invention relates to a microarray useful to distinguish among subtypes of RCCs, comprising a matrix of at least one cDNA probe from one or more sets of probes immobilized to a solid surface in predetermined order such that a row of pixels corresponds to replicates of one distinct probe from one of the sets, the probes being any of a set represented by SEQ ID NOs:1-30; a set represented by SEQ ID NOs: 31-60; a set represented by SEQ ID NOs:61-90; a set represented by SEQ ID NOs:91-93; a set represented by SEQ ID NOs: 94-98; and/or a set represented by SEQ ID NOs:99-100,

wherein the probes in each set are complementary to nucleic acid sequences expressed differentially in different subtypes of renal cell carcinomas (RCC), which nucleic acid sequences hybridize to the probes under high stringency conditions.

For analysis of the target nucleic acid of primary tumor tissue, the preferred analyte of this invention is isolated from tissue biopsies before they are stored or from fresh-frozen tumor tissue of the primary tumor which may be stored and/or cultured in standard culture media. For expression studies, poly(A)-containing mRNA is isolated using commercially available kits, e.g., from Invitrogen, Oligotex, or Qiagen. The isolated mRNA is assayed directly or, preferably, is reverse transcribed into cDNA in the presence of a labeled nucleotides. Fluorescent cDNA is generally synthesized using reverse transcriptase (e.g., Superscript II reverse-transcription kit from GIBCO-BRL) and nucleotides to which is conjugated a fluorescent label. A preferred fluorescent label is Cy5 conjugated to dUTP and/or dCTP (from Amersham). Additional, optional, methods of amplification of the target, such as by PCR, are also included in the methods of the invention.

In one embodiment, the present method employs immobilized cDNA probes of anywhere between about 15 bases up to a fall length cDNA, e.g., about 2000 bases. Preferred probes have about 100 bases. Optimal hybridization conditions (temperature, pH, ion and salt concentrations, and incubation time) are dependent on the length of the shortest probes as the limiting step and can be adjusted in a continuous fashion by varying the above parameters as is conventional in the art. In a preferred embodiment, probes of the invention hybridize specifically to target polynucleotides of interest under conditions of high stringency. As used herein, “conditions of high stringency” or “high stringent hybridization conditions” means any conditions in which hybridization will occur when there is at least about 95%, preferably about 97 to 100%, nucleotide complementarity (identity) between the nucleic acids (e.g., a polynucleotide of interest and a nucleic acid probe). However, depending on the desired purpose, hybridization conditions can be selected which require less complementarity, e.g., about 90%, 85%, 75%, 50%, etc. Appropriate hybridization conditions include, e.g., hybridization in a buffer such as, for example, 6×SSPE-T (0.9 M NaCl, 60 mM NaH₂ PO₄, 6 mM EDTA and 0.05% Triton X-100) for between about 10 minutes and about at least 3 hours (in a preferred embodiment, at least about 15 minutes) at a temperature ranging from about 4° C. to about 37° C.

Several probe sequences described herein are cDNAs complementary to genes or gene fragments; some are ESTs. Those skilled in the art will appreciate that a probe of choice for a particular gene can be the full length coding sequence or any fragment thereof having generally at least about 8 or at least about 15 nucleotides. Thus, when the fall length sequence is known, the practitioner can select any appropriate fragment of that sequence. When the original results are obtained using partial sequence information (e.g., an EST probe), and when the full length sequence of which that EST is a fragment becomes available (e.g., in a genome database), the skilled artisan can select a longer fragment than the initial EST, as long as the length is at least about 8 or at least about 15 nucleotides.

The arrays of the present invention comprise one or more nucleic acid probes having hybridizable fragments of any length (from about 15 bases to full coding sequence) for the genes whose expression is to be analyzed. For purposes of the analysis, it is not necessary that the full length sequence be known, as those of skill in the art will know how to obtain the full length sequences using the sequence of a given EST and known data mining, bioinformatics, and DNA sequencing methodologies without undue experimentation.

The nucleic acid probes of the present invention may be native DNA or RNA molecules or analogues of DNA or RNA. The present invention is not limited to the use of any particular DNA or RNA analogue; rather any one is useful provided that it is capable of adequate hybridization to a complementary DNA strand (or mRNA) in a test sample, has adequate resistance to nucleases and stability in the hybridization protocols employed. DNA or RNA may be made more resistant to nuclease degradation in vivo by modifying internucleoside linkages (e.g., methylphosphonates or phosphorothioates) or by incorporating modified nucleosides (e.g., 2′-0-methylribose or 1′-α-anomers) as described below.

A nucleic acid may comprise at least one modified base moiety, for example, 5-fluorouracil, 5-bromouracil, 5-chlorouracil, 5-iodouracil, hypoxanlthine, xanthine, 4-acetylcytosine, 5-(carboxyhydroxylmethyl)uracil, 5-carboxymethylaminomethyl-ω-thiouridine, 5-carboxymethyl-aminomethyl uracil, dihydrouracil, β-D-galactosylqueosine, inosine, N6-isopentenyladenine, 1-methylguanine, 3-methyl-cytosine, 5-methylcytosine, N6-adenine, 7-methylguanine, 5-methylaminomethyluracil, 5-methoxyamino-methyl-2-thiouracil, β-D-mannosylqueosine, 5-methoxy-carboxymethyluracil, 5-methoxyuracil-2-methylthio-N-6-iso-pentenyladenine, uracil-5-oxyacetic acid, butoxosine, pseudouracil, queuosine, 2-thio-cytosine, 5-methyl-2-thiouracil, 2-thiouracil, 4-thiouracil, 5-methyluracil, uracil-5-oxyacetic acid methylester, uracil-t-oxyacetic acid, 5-methyl-2-thiouracil, 3(3-amino-3-N-2-carboxypropyl) uracil and 2,6-diaminopurine.

The nucleic acid may comprise at least one modified sugar moiety including, but not limited, to arabinose, 2-fluoroarabinose, xylulose, and hexose.

In yet another embodiment, the nucleic acid probe comprises a modified phosphate backbone synthesized from a nucleotide having, for example, one of the following structures: a phosphorothioate, a phosphoridothioate, a phosphoramidothioate, a phosphoramidate, a phosphordiimidate, a methylsphosphonate, an alkyl phosphotriester, 3′-aminopropyl and a formacetal or analog thereof.

In yet another embodiment, the nucleic acid probe is an α-anomeric oligonucleotide which forms specific double-stranded hybrids with complementary RNA in which, contrary to the usual β-units, the strands run parallel to each other (Gautier et al., 1987, Nucl. Acids Res. 15:6625-6641).

A nucleic acid probe (e.g., an oligonucleotide) may be conjugated to another molecule, e.g., a peptide, a hybridization-triggered cross-linking agent, a hybridization-triggered cleavage agent, etc., all of which are well-known in the art.

Nucleic acid probes (e.g., oligonucleotides) of this invention may be synthesized by standard methods known in the art for example, by using an automated DNA synthesizer (such as those are commercially available from Biosearch, Applied Biosystems, etc.). As examples, phosphorothioate oligonucleotides may be synthesized by the method of Stein et al., Nucl. Acids Res. (1998) 16:3209, methylphosphonate oligonucleotides can be prepared by use of controlled pore glass polymer supports (Sarin et al., Proc. Natl. Acad. Sci. U.S.A. (1988) 85:7448-7451), etc.

The invention also relates to probe molecules that are at least about 75% identical to a polynucleotide target of interest, or at least about 80%, 90%, 95% or 99% complementary thereto. Conventional algorithms can be used to determine the percent complementarity, e.g., as described by Lipman and Pearson (Proc. Natl. Acad Sci 80:726-730, 1983) or Martinez/Needleman-Wunsch (Nuci Acid Research 11:4629-4634, 1983).

Nucleic acids of the invention may be detected by any of a variety of conventional methods. Preferred detectable labels include a radionuclides, fluorescers, fluorogens, a chromophore, a chromogen, a phosphorescer, a chemiluminescer or a bioluminescer. Examples of fluorescers or fluorogens are i fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green or Texas Red.

Common fluorescent labels include fluorescein, rhodamine, dansyl, phycoerythrin, phycocyanin, allophycocyanin, o-phthaldehyde and fluorescamine. Most preferred are the labels described in the Examples, below.

The fluorophore must be excited by light of a particular wavelength to fluoresce. See, for example, Haugland, Handbook of Fluorescent Probes and Research Chemicals, Sixth Ed., Molecular Probes, Eugene, Oreg., 1996).

Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Similarly, fluorophores may also be coupled to thiols using maleimide, iodoacetamide, and aziridine-reactive groups. The long wavelength rhodamines, which are basically Rhodamine Green™ derivatives with substituents on the nitrogens, are among the most photostable fluorescent labeling reagents known. Their spectra are not affected by changes in pH between 4 and 10, an important advantage over the fluoresceins for many biological applications. This group includes the tetramethylrhodamines, X-rhodamines and Texas Red™ derivatives. Other preferred fluorophores are those which are excited by ultraviolet light. Examples include cascade blue, coumarin derivatives, naphthalenes (of which dansyl chloride is a member), pyrenes and pyridyloxazole derivatives.

The present invention serves as a basis for even broader implementation of arrays, such as microarrays, and gene expression in deducing important pathways implicated in the different subtypes of renal cancer. For example, the expression patterns disclosed herein are based on an analysis of about 70 kidney tumors. As additional patient samples are analyzed, larger databases may be generated that provide even more information concerning metabolic differences among the various types of renal cancers. Correlations with other factors, such as clinical outcome, can add even further understanding.

Other aspects of the invention relate to methods to determine the subtype of an RCC in a subject, comprising detecting the presence of, and/or quantitating the amount of, one or more protein products whose expression is upregulated in a majority of subjects suffering from one of the subtypes of RCC as discussed elsewhere herein. The terms “protein” and “polypeptide” are used interchangeably herein.

Examples of such proteins are those discussed above as components of protein-containing compositions of the invention. The protein can be, e.g., a secreted protein, an intracellular protein which is rendered accessible by permeabilizing the cell in which it resides, or a cell surface expressed protein. The presence or quantity of the protein product in a body fluid or, preferably, in a tissue or cell sample from the kidney of the subject, is determined. An increased level of the protein product compared to the level in a normal subject's fluid, or in a normal (noncancerous) kidney sample from the subject or from a reference normal value (e.g., from pool of normal subjects), is indicative of the presence of a particular subtype of renal cell carcinoma. Proteins whose overexpression are indicative of particular subtypes of RCC are discussed elsewhere herein.

Methods of preparing patient samples, such as kidney samples, and detecting and/or quantitating proteins therein are conventional and well known in the art. Some such methods are discussed elsewhere herein.

In a particularly preferred method, the proteins are detected by immunological methods, such as, e.g., immunoassays (EIA), radioimmunoassay (RIA), immunofluorescence microscopy, or immunohistochemistry, all of which assay methods are fully conventional.

Any of a variety of antibodies can be used in such methods. Such antibodies include, e.g., polyclonal, monoclonal (imAbs), recombinant, humanized or partially humanized, single chain, Fab, and fragments thereof. The antibodies can be of any isotype, e.g., IgM, various IgG isotypes such as IgG_{1′}IgG₂ₐ, etc., and they can be from any animal species that produces antibodies, including goat, rabbit, mouse, chicken or the like. An antibody “specific for” a polypeptide means that the antibody recognizes a defined sequence of amino acids, or epitope, either present in the full length polypeptide or in a peptide fragment thereof. Antibodies can be prepared according to conventional methods, which are well known. See, e.g., Green et al., Production of Polyclonal Antisera, in Immunochemical Protocols (Manson, ed.), (Humana Press 1992); Coligan et al., in Current Protocols in Immunology, Sec. 2.4.1 (1992); Kohler & Milstein, Nature 256:495 (1975); Coligan et al., sections 2.5.1-2.6.7; and Harlow et al., Antibodies: A Laboratory Manual, page 726 (Cold Spring Harbor Laboratory Pub. 1988). Methods of preparing humanized or partially humanized antibodies, and antibody fragments, and methods of purifying antibodies, are conventional

Determination of optimal concentrations of antibodies for use in immunohistochemical techniques is accomplished using standard methods, i.e., titrating a test antibody against an appropriate tissue sample. As is known the art, antibody preparations are commonly used at higher concentrations for immunohistochemistry than in EIAs and other such immunoassays.

The molecular profiling information described herein can also be harnessed for the purpose of discovering drugs that are selected for their ability to correct or bypass the molecular alterations or derangements that are characteristic of the various renal carcinoma sub-types described herein. A number of approaches are available.

In one embodiment, RCC cell lines are prepared from tumors using standard methods and are profiled using the present methods. Preferred cell lines are those that maintain the expression profile of the primary tumor from which they were derived. One or several RCC cell lines may be used as a “general” panel; alternatively or additionally, cell lines from individual subjects may be prepared and used. These cell lines are used to screen compounds, preferably by high-throughput screening (HTS) methods, for their ability to alter the expression of selected genes. Typically, small molecule libraries available from various commercial sources are tested by HTS protocols.

The molecular alterations in the cell line cells can be measured at the mRNA level (gene expression) applying the methods disclosed in detail herein. Alternatively, one may assay the protein product(s) of the selected gene(s). Thus, in the case of secreted or cell-surface proteins, expression can be assessed using immunoassay or other immunological methods including enzyme immunoassays (EIA), radioimmunoassay (RIA), immunofluorescence microscopy or flow cytometry. EIAs are described in greater detail in several references (Butler, J E, In: Structure of Antigens, Vol. 1 (Van Regenmortel, M., CRC Press, Boca Raton 1992, pp. 209-259; Butler, J E, “ELISA,” In: van Oss, C. J. et al. (eds), Immunochemistry, Marcel Dekker, Inc., New York, 1994, pp. 759-803; Butler, J E (ed.), Immunochemistry of Solid-Phase Immunoassay, CRC Press, Boca Raton, 1991). RIAs are discussed in Kirkham and Hunter (eds.), Radioimmune Assay Methods, E. & S. Livingstone, Edinburgh, 1970.

In another approach, antisense RNAs or DNAs that specifically inhibit the transcription and/or translation of the targeted genes can be screened for specificity and efficacy using the present methods. Antisense compositions would be particularly useful for treating tumors in which a particular gene is up-regulated (e.g., the genes in Tables 1, 2, 3, 5 and 6, or the genes identified for Wilms Tumor).

The protein products of genes that are upregulated in most cases of the renal tumors described herein (Tables 1, 2, 3, 5 and 6, and the two genes identified for Wilms' tumor) are targets for diagnostic assays if the proteins can be detected by some assay means, e.g., immunoassay, in some accessible body fluid or tissue.

One class of diagnostic targets is secreted proteins which reach a measurable level in a body. Thus, a sample of a body fluid such as such as plasma, serum, urine, saliva, cerebrospinal fluid, etc., is obtained from the subject being screened. The sample is subject to any known assay for the protein analyte. Alternatively, cells expressing the protein on their surface may be obtained, e.g., blood cells, by simple, conventional means. If the protein is a receptor or other cell surface structure, it can be detected and quantified by well-known methods such as flow cytometry, immunofluorescence, immunocytochemistry or immunohistochemistry, and the like.

In a preferred embodiment, diagnosis is performed on a sample from a kidney tumor, e.g., a biopsy tissue, a fresh-frozen sample, or, in a most preferred embodiment, a section of a paraffin-embedded block of tissue. Methods of preparing all of these sample types are conventional and well known in the art. Biopsy material and fresh-frozen samples can be extracted by conventional procedures to obtain proteins or polypeptides therein. In one embodiment, paraffin-embedded blocks are sectioned and analyzed directly without such extractions. An example showing immunohistochemical analysis of such paraffin blocks is shown in Example 1 and FIG. 3.

Preferably, an antibody or other protein or peptide ligand for the target protein to be detected is used. In another embodiment where the gene product is a receptor, a peptidic or small molecule ligand for the receptor may be used in known assays as the basis for detection and quantitation.

In vivo methods with appropriately labeled binding partners for the protein targets, preferably antibodies, may also be used for diagnosis and prognosis, for example to image occult metastatic foci or for other types of in situ evaluations. These methods utilize include various radiographic, scintigraphic and other imaging methods well-known in the art (MRI, PET, etc.).

Suitable detectable labels include radioactive, fluorescent, fluorogenic, chromogenic, or other chemical labels. Useful radiolabels, which are detected simply by gamma counter, scintillation counter or autoradiography include ³H, ¹²⁵I, ¹³¹I, ³⁵S and ¹⁴C.

Common fluorescent labels include fluorescein, rhodamine, dansyl, phycoerythrin, phycocyanin, allophycocyanin, o-phthaldehyde and fluorescamine. The fluorophore, such as the dansyl group, must be excited by light of a particular wavelength to fluoresce. See, Haugland, Handbook of Fluorescent Probes and Research Chemicals, Sixth Ed., Molecular Probes, Eugene, Oreg., 1996). Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Fluorophores may also be coupled to thiols using maleimide, iodoacetamide, and aziridine-reactive groups. The long wavelength rhodamines include the tetramethylrhodamines, X-rhodamines and Texas Red™ derivatives. Other preferred fluorophores for derivatizing the protein binding partner are those which are excited by ultraviolet light. Examples include cascade blue, coumarin derivatives, naphthalenes (of which dansyl chloride is a member), pyrenes and pyridyloxazole derivatives.

The protein (antibody or other ligand) can also be labeled for detection using fluorescence-emitting metals such as ¹⁵²Eu, or others of the lanthanide series. These metals can be attached to the protein using metal chelating groups such as diethylenetriaminepentaacetic acid (DTPA) or ethylenediaminetetraacetic acid (EDTA).

For in vivo diagnosis, radionuclides may be bound to protein either directly or indirectly using a chelating agent such as DTPA and EDTA which is chemically conjugated, coupled or bound (which terms are used interchangeably) to the protein. The chemistry of chelation is well known in the art. The key limiting factor on the chemistry of coupling is that the antibody or ligand must retain its ability to bind the target protein. A number of references disclose methods and compositions for complexing metals to macromolecules including description of useful chelating agents. The metals are preferably detectable metal atoms, including radionuclides, and are complexed to proteins and other molecules. See, for example, U.S. Pat. Nos. 5,627,286, 5,618,513, 5,567,408, 5,443,816 and 5,561,220, all of which are incorporated by reference herein.

Any radionuclide having diagnostic (or therapeutic value) can be used. In a preferred embodiment, the radionuclide is a γ-emitting or β-emitting radionuclide, for example, one selected from the lanthanide or actinide series of the elements. Positron-emitting radionuclides, e.g. ⁶⁸Ga or ⁶⁴Cu, may also be used. Suitable β-emitting radionuclides include those which are useful in diagnostic imaging applications. The gamma-emitting radionuclides preferably have a half-life of from 1 hour to 40 days, preferably from 12 hours to 3 days. Examples of suitable γ-emitting radionuclides include ⁶⁷Ga, ¹¹¹In, ^{99m}Tc, ¹⁶⁹Yb and ¹⁸⁶Re. Examples of preferred radionuclides (ordered by atomic number) are ⁶⁷Cu, ⁶⁷Ga, ⁶⁸Ga, ⁷²As, ⁸⁹Zr, ⁹⁰Y, ⁹⁷Ru, ⁹⁹Tc, 111In, ¹²³I, ¹²⁵I, ¹³¹I, ¹⁶⁹Yb, ¹⁸⁶Re, and ²⁰¹Tl. Though limited work have been done with positron-emitting radiometals as labels, certain proteins, such as transferrin and human serum albumin, have been labeled with ⁶⁸Ga,

A number of metals (not radioisotopes) useful for MRI include gadolinium, manganese, copper, iron, gold and europium. Gadolinium is most preferred. Dosage can vary from 0.01 mg/kg to 100 mg/kg.

In situ detection of the labeled protein may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

The compositions of the present invention may be used in diagnostic, prognostic or research procedures in conjunction with any appropriate cell, tissue, organ or biological sample of the desired animal species. By the term “biological sample” is intended any fluid or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term is a organ or tissue extract and a culture fluid in which any cells or tissue preparation from the subject has been incubated. Samples from renal tissue are preferred.

An alternative diagnostic approach utilizes cDNA probes that are complementary to and thereby detect cells in which a gene associated with a subtype of RCC is upregulated by in situ hybridization with mRNA in these cells. The present invention provides methods for localizing target mRNA in cells using fluorescent in situ hybridization (FISH) with labeled cDNA probes having a sequence that hybridizes with the mRNA of an upregulated gene. The basic principle of FISH is that DNA or RNA in the prepared specimens are hybridized with the probe nucleic acid that is labeled non-isotopically with, for example, a fluorescent dye, biotin or digoxigenin. The hybridized signals are then detected by fluorimetric or by enzymatic methods, for example, by using a fluorescence or light microscope. The detected signal and image can be recorded on light sensitive film.

An advantage of using a fluorescent probe is that the hybridized image can be readily analyzed using a powerful confocal microscope or an appropriate image analysis system with a charge-coupled device (CCD) camera. As compared with radioactive methods, FISH offers increased sensitivity. In additional to offering positional information, FISH allows better observation of cell or tissue morphology. Because of the nonradioactive approach, FISH has become widely used for localization of specific DNA or mRNA in a specific cell or tissue type.

The in situ hybridization methods and the preparations useful herein are describe in Wu, W. et al., eds., Methods in Gene Biotechnology, CRC Press, 1997, chapter 13, pages 279-289. This book is incorporated by reference in its entirety, as are the references cited therein. A number of patents and papers that describe various in situ hybridization techniques and applications, also incorporated by reference, are: U.S. Pat. Nos. 5,912,165; 5,906,919; 5,885,531; 5,880,473; 5,871,932; 5,856,097; 5,837,443; 5,817,462; 5,784,162; 5,783,387; 5,750,340; 5,759,781; 5,707,797; 5,677,130; 5,665,540; 5,571,673; 5,565,322; 5,545,524; 5,538,869; 5,501,954, 5,225,326, and 4,888,278. Other related references include Jowett, T, Methods Cell Biol; 59:63-85 (1999) Pinkel et al., Cold Spring Harbor Symp. Quant. Biol. LI:151-157 (1986); Pinkel, D. et al., Proc. Natl. Acad. Sci. (USA) 83:2934-2938 (1986); Gibson et al., Nucl. Acids Res. 15:6455-6467 (1987); Urdea et al., Nucl. Acids Res. 16:4937-4956 (1988); Cook et al., Nucl. Acids Res. 16:4077-4095 (1988); Telser et al., J. Am. Chem. Soc. 111:6966-6976 (1989); Allen et al., Biochemistry 28:4601-4607 (1989); Nederlof, P. M. et al., Cytometry 10:20-27 (1989); Nederlof, P. M. et al., Cytometry 11:126-131 (1990); Seibl, R., et al., Biol. Chem. Hoppe-Seyler 371:939-951 (October 1990); Wiegant, J. et al., Nucl. Acids Res. 19:3237-3241 (1991); McNeil J A et al., Genet Anal Tech Appl 8:41-58 (1991); Komminoth et al., Diagnostic Molecular Biology 1:85-87 (1992); Dauwerse, J G et al., Hum. Mol. Genet. 1:593-598 (1992); Ried, T. et al., Proc. Natl. Acad. Sci. (USA) 89:1388-1392 (1992); Wiegant, J. et al., Cytogenet. Cell Genet. 63:73-76 (1993); Glaser, V., Genetic. Eng. News. 16:1, 26 (1996); Speicher, M R, Nature Genet. 12:368-375 (1996).

In a case in which an upregulated gene, e.g., DNA sequence “X” is identified but its protein product “Y” is unknown, one would first examine the expressed DNA sequence X. The full length gene sequence may be obtained by accessing a human genomic database such as that of Celera. In either case, examination of the coding sequence for appropriate motifs will indicate whether the encoded protein Y is secreted protein or a transmembrane protein. If no antibodies specific for protein Y are already available, peptides of protein Y can be designed and synthesized using known principles of protein chemistry and immunology. The object is to create a set of immunogenic peptides that elicit antibodies specific for surface epitopes of the protein. Alternatively, the coding DNA or portions thereof can be expression-cloned to produce a polypeptide or a peptide thereof. That protein or peptide can be used as an immunogen to immunize animals for the production of antisera or to prepare mAbs. These polyclonal sera or mAbs can then be applied in an immunoassay, preferably an EIA, to detect the presence of protein Y or measure its concentration in a body fluid or cell/tissue sample.

Taking the lead from the drug discovery methods described above, one can exploit the present invention to treat kidney tumors based on the knowledge of the genes that are upregulated in a highly predicable manner in any particular renal tumor subtype. (see Tables 1-3, 5, and 6). Based on the nature of the deduced protein product, one can devise a means to inhibit the action of, or bind, block, remove or otherwise diminish the presence and availability of the upregulated protein. In the case of a cellular receptor, one would expose the upregulated receptor to an antagonist, a soluble form of the receptor or a “decoy” ligand binding site of a receptor (to compete for ligand) (Gershoni J M et al., Proc Natl Acad Sci USA, 1988, 85:4087-9; U.S. Pat. No. 5,770,572).

Antibodies may be administered to a subject to bind and inactivate (or compete with) secreted protein products or expressed cell-surface products of upregulated genes.

Another therapeutic approach is to employ antisense oligonucleotide or polynucleotide constructs that inhibit gene expression of an upregulated gene in a highly specific manner. Methods to select, test and optimize putative antisense sequences are routine, as are methods to operatively link appropriate antisense sequences to an appropriate regulatory element, e.g., a promoter, such as a strong promoter, an inducible strong promoter, or the like. Inducible promoters include, e.g., an estrogen inducible system (Braselmann, S. et al Proc Natl Acad Sci USA (1993) 90:1657-1661). Also known are repressible systems driven by the conventional antibiotic, tetracycline (Gossen, M. et al., Proc. Natl. Acad. Sci. USA 89:5547-5551 (1992)). Multiple antisense constructs specific for different upregulated genes can be employed together. The sequences of the upregulated genes described herein can be used to design the antisense oligonucleotides (Hambor, J E et al., J. Exp. Med. 168:1237-1245 (1988); Holt, J T et al., Proc. Nat'l. Acad. Sci. 83:4794-4798 (1986); Izant, J G et al., Cell 36:1007-1015 (1984); Izant, J G et al, Science 229:345-352 (1985); De Benedetti, A. et al, Proc. Natl. Acad. Sci. USA, 84:658-662 (1987)). The antisense oligonucleotides may range from about 6 to about 50 nucleotides, and may be as large as 100 or 200 nucleotides, or larger. The oligonucleotides can be DNA or RNA or chimeric mixtures or derivatives or modified versions thereof, single-stranded or double-stranded. The oligonucleotides can be modified at the base moiety, sugar moiety, or phosphate backbone (as discussed above). The oligonucleotide may include other appending groups such as peptides, or agents facilitating transport across the cell membrane (see, e.g. Letsinger et al., 1989, Proc. Natl. Acad. Sci. USA 84:684-652; PCT Publication WO 88/09810 (1988) or blood-brain barrier (e.g., PCT Publication WO 89/10134 (1988), hybridization-triggered cleavage agents (e.g. Krol et al, 1988, BioTechniques 6:958-976) or intercalating agents (e.g., Zon, 1988, Pharm. Res 5:539-549). Other therapeutic methods, such as the use of ribozymes that can specifically cleave nucleic acids encoding the overexpressed genes of the invention are also contemplated by the invention. Such methods are routine in the art and methods of making and using any of a variety of appropriate ribozymes are well known to the skilled worker.

Another therapeutic approach involves double stranded RNAs called small interfering RNA (RNAi). RNAi molecules can be used to inhibit gene expression, using conventional procedures. Typical methods to make and use interfering RNA molecules are described, e.g., in U.S. Pat. No. 6,506,559.

Methods of gene transfer can be used, wherein oligonucleotides such antisense molecules or ribozymes are introduced into a renal tumor cell or tissue or other tissue or organ of interest, or nucleic acids that encode proteins which interfere with the production or activity of one or more of the overexpressed genes of the invention are so introduced. Therapeutic methods that require gene transfer and targeting may include virus-mediated gene transfer, for example, with retroviruses (Nabel, E. G. et al., Science 244:1342 (1989), lentiviruses, recombinant adenovirus vectors (Horowitz, M. S., In: Virology, Fields, B N et al., eds, Raven Press, New York, 1990, p. 1679, or current edition; Berkner, K L, Biotechniques 6:616 919, 1988), Strauss, SE, In: The Adenoviruses, Ginsberg, HS, ed., Plenum Press, New York, 1984, or current edition), Adeno-associated virus (AAV) is also useful for human gene therapy (Samulski, R J et al., EMBO J. 10:3941 (1991); (Lebkowski, J S, et al., Mol. Cell. Biol. (1988) 8:3988-3996; Kotin, R M et al., Proc. Natl. Acad. Sci. USA (1990) 87:2211-2215); Hermonat, P L, et al., J. Virol. (1984) 51:329-339). Improved efficiency is attained by the use of promoter enhancer elements in the plasmid DNA constructs (Philip, R. et al, J. Biol. Chem. (1993) 268:16087-16090).

In addition to virus-mediated gene transfer in vivo, physical means well-known in the art can be used for direct gene transfer, including administration of plasmid DNA (Wolff et al., 1990, supra) and particle-bombardment mediated gene transfer, originally described in the transformation of plant tissue (Klein, T M et al., Nature 327:70 (1987); Christou, P. et al., Trends Biotechnol. 6:145 (1990)) but also applicable to mammalian tissues in vivo, exk vivo or in vitro (Yang, N.-S., et al., Proc. Natl. Acad. Sci. USA 87:9568 (1990); Williams, R S et al., Proc. Natl. Acad. Sci. USA 88:2726 (1991); Zelenin, A V et al., FEBS Lett. 280:94 (1991); Zelenin, A V et al., FEBS Lett. 244:65 (1989); Johnston, S. A. et al., In Vitro Cell. Dev. Biol. 27:11 (1991)). Furthermore, electroporation, a well-known means to transfer genes into cell in vitro, can be used to transfer DNA molecules according to the present invention to tissues in vivo (Titomirov, A V et al., Biochim. Biophys. Acta 1088:131 ((1991)).

Gene transfer can also be achieved using “carrier mediated gene transfer” (Wu, C H et al., J. Biol. Chem. 264:16985 (1989); Wu, G Y et al., J. Biol. Chem. 263:14621 (1988); Soriano, P et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc. Natl. Acad. Sci. USA 80:1068 (1983); Soriano et al., supra) such as immunoliposomes, which can incorporate acylated monoclonal antibodies into the lipid bilayer (Wang et al., supra), or polycations such as asialoglycoprotein/polylysine (Wu et al., 1989, supra). Liposomes have been used to encapsulate and deliver a variety of materials to cells, including nucleic acids and viral particles (Faller, D V et al., J. Virol. (1984) 49:269-272).

Preformed liposomes that contain synthetic cationic lipids form stable complexes with polyanionic DNA (Felgner, P L, et al., Proc. Natl. Acad. Sci. USA (1987) 84:7413-7417). Cationic liposomes, liposomes comprising some cationic lipid, that contained a membrane fusion-promoting lipid dioctadecyldimethyl-ammonium-bromide (DDAB) have efficiently transferred heterologous genes into eukaryotic cells (Rose, J K et al., Biotechniques (1991) 10:520-525). Cationic liposomes can mediate high level cellular expression of transgenes, or mRNA, by delivering them into a variety of cultured cell lines (Malone, R, et al., Proc. Natl. Acad. Sci. USA (1989) 86:6077-6081).

One can also exploit the present invention to monitor the treatment of kidney tumors, based on the knowledge of the genes that are upregulated in a highly predicable manner in any particular renal tumor subtype. At various stages during the course of the treatment of a subject, renal samples may be taken and prepared for analysis, as described elsewhere herein, and analyzed for the presence and/or amount of one or more the upregulated genes whose overexpression correlates with the type of renal tumor being treated, compared to the amount in a normal renal tissue. Successful treatment will be reflected by a change in the expression pattern to one more closely resembling that of a normal renal tissue.

The present invention also relates to combinations of nucleic acids or polypeptides of the invention represented, not by physical molecules, but by computer-implemented databases that list or otherwise include or represent these sequences, etc. For example, the present invention includes electronic forms of information representing the polynucleotides, polypeptides, etc., of the present invention, including the computer-readable medium (e.g., magnetic, optical, etc.) on which this information is stored in any suitable format, such as flat files or hierarchical files. This information preferably comprises full length or partial sequences and e-commerce-type means for manipulating, retrieving, and sharing the information, etc. For example, an investigator may compare an expression profile exhibited by a renal carcinoma sample of interest to data in an electronic or other computer-readable form that describes or represents a compositions of the invention, and may thereby determine the subtype of the renal tumors being evaluated.

Having now generally described the invention, the same will be more readily understood through reference to the following examples which are provided by way of illustration, and are not intended to be limiting of the present invention, unless specified.

### EXAMPLE I

Subjects and Tumor Samples

A total of 69 frozen primary kidney tumors (39 clear cell RCC, 7 papillary RCC, 6 granular RCC, 5 chromophobe RCC, 2 sarcomatoid RCC, 2 oncocytomas, 3 TCCs, and 5 Wilms' tumors), 1 metastatic papillary RCC and matched or unmatched noncancerous kidney tissue were obtained from the University of Tokushima, the University of Chicago, Spectrum Health Urologic Group and Cooperative Human Tissue Network (CHTN). All tissues were accompanied by pathology reports with or without clinical outcome information. The samples were anonymized prior to the study. Part of each tumor sample was frozen in liquid nitrogen immediately after surgery and stored at −80° C.

Conventional methods were used for nucleic acid isolation and preparation. Total RNA was isolated from the frozen tissues using ISOGEN solution (Nippon Gene, Toyama, Japan) or Trizol reagent (Invitrogen, Carlsbad, Calif.). For the first 45 samples, poly(A)+ RNA was isolated from the total RNA using the Oligotex mRNA Mini Kit (Qiagen, Valencia, Calif.). For the remaining 25 samples, total RNA was purified with 2.5 M final concentration of LiCl. The WHO International Histological Classification of Tumors was used for histological evaluation of the specimens (Mostfi, 1998 supra). UICC (Union Internationale Contre le Cancer) TNM classification and stage groupings were used (Sobin et al., editors, International Union Against Cancer. 5^{th} edition. New York: John Wiley & Sons, 1997).

### EXAMPLE II

### Materials and Methods

Microarray Design and Procedures

Microarrays were produced using conventional methods and materials well known in the art (Hegde et al., Biotechniques 2000; 29:548-556; Eisen et al., Methods Enzymol (1999) 303:179-205) with slight modifications. Bacterial libraries purchased from Research Genetics, Inc. were the source of 19,968 cDNAs which were PCR amplified directly. cDNA clones were ethanol-precipitated and transferred to 384-well plates from which they were printed onto aminosilane coated glass slides using a home-built robotic microarrayer (see, e.g., the web site at microarrays.org/pdfs/PrintingArrays. Slides were chemically blocked using succinic anhydrate after UV crosslinking. When available, cancers were hybridized against patient matched non-cancerous kidney tissue. For tumors without their matched noncancerous kidney tissue available, RNA from five noncancerous kidney tissues was mixed and pooled for serving as a common reference. For the first 45 samples, two μg of poly(A)+ RNA from tumors and reference were reverse transcribed with oligo (dT) primer and Superscript II (Invitrogen, Carlsbad, Calif.) in the presence of Cy5-dCTP and Cy3-dCTP (Amershamn Pharmacia Biotech, Peapack, N.J.). For the remaining 25 samples, 50 μg of total RNA from tumors and reference were used for reverse transcription. The Cy5- and Cy3-labeled cDNA probes were mixed with probe hybridization solution containing formamide and hybridized to pre-warmed (50° C.) slides for 20 hours at 50° C. Following hybridization, slides were washed in 1×SSC, 0.1% SDS at 50° C. for 5 minutes followed by 0.2×SSC, 0.1% SDS at room temperature (RT) for 5 minutes, 0.2×SSC at RT for 5 minutes twice, and 0.1×SSC at RT for 5 minutes. Slides were dried immediately by centrifugation and scanned using a Scan Array Lite scanner at 532 nm and 635 nm wavelengths (GSI Lumonics, Billerica, Calif.).

Data Analysis

Images were analyzed using the software Genepix Pro 3.0 (Axon, Union City, Calif.). The local background was subtracted for all spots. Spots whose background-subtracted intensities in either Cy5 or Cy3 channel were less than 150 were excluded from the analysis. The ratio of Cy5 intensity to Cy3 intensity was calculated for each spot, representing tumor RNA expression relative to noncancerous kidney tissue. Ratios were log transformed (base 2) and normalized so that the median log-transformed ratio equaled zero. Genes with the following criteria (3560 genes in total) were selected for the global clustering analysis: 1) expression values present in at least 70% of the tumors; 2) expression ratios that varied at least two-fold in at least two tumors; and 3) maximum ratio minus minimum ratio values greater than two-fold. The gene expression ratios were median polished across all samples. Gene expression values were manipulated and visualized using the CLUSTER and TREEVIEW software (M. B. Eisen, available at the website having the URL rana.lbl.gov). The correlation distances were calculated as 1-r, where r indicates the Pearson rank correlation coefficient (Eisen et al., Proc Natl Acad Sci USA 1998, 95:14863-14868).

The in-house software program, CIT, was used to find genes that were differentially expressed (using a student's t-test) between one histological subtype and the others (Rhodes et al., Bioinformatics 2002, 18:205-206). To find significant discriminating genes, 10,000 t-statistics were calculated by randomly placing patients into two groups (Hedenfalk et al., 2001, supra). A 99.9% significance threshold (p<0.01) was used to identify genes that could significantly distinguish between two patient groups versus the random patient groupings.

The clustering analysis of the 70 kidney tumors was displayed as follows: The clustering of patients (using Pearson's correlation) was based on global gene expression profiles consisting of median polished data of 3,560 selected spots. Rows represented individual cDNAs and columns represented individual tumor samples. The color of each square represented the median-polished, normalized ratio of gene expression in a tumor relative to reference. Expression levels greater than the median were indicated with different colors. The color saturation indicated the degree of divergence from the median. The tumors clustered into two broad groups with one group consisting of primarily clear cell RCC and the other consisting of all other kidney tumors. Five chromophobe RCC and two oncocytoma were clustered close together. Each group of eight papillary RCC, five Wilms tumors, or three TCC was clustered together. A set of the most highly expressed genes in each subtype of tumors compared to all other types of kidney tumors studied was identified.

The data were also displayed as three-dimensional (3D) tumor images. Various subtypes of kidney tumor were each represented by different colors. Five chromophobe RCC and two oncocytoma clustered close together. The eight papillary RCC, five Wilms tumors, and three TCC clustered close together respectively. Clear cell RCC on the other hand looked more scattered than in 2D clustering by TreeView. All tumors with a focus on CC-RCC whose outcome data were available were displayed. Patients who survived more than five years after surgery, and patients who died of cancer within five years after surgery, were represented by different colors.

Immunohistochemistry

Fifty renal tissue samples, both benign (n—10) and neoplastic (n=40) were analyzed using immunohistochemistry. Kidney tumors included clear cell RCC (n=10), papillary RCC (n=10), chromophobe RCC (n=10), oncocytoma (n-5) and TCC (n=5). A section from each tissue sample was stained with hematoxylin and eosin to verify histology. Antibodies to the following proteins were obtained commercially: GSTα, a methylacyl racemate (Corixa, Seattle, Wash., USA), carbonic anhydrase II and keratin 19 (Dako, Carpinteria, Calif., USA). Standard biotin-avidin-complex immunohistochemistry was performed. Briefly, tissue sections were incubated with primary antibodies for 30 min. at 20° C. Then, the slides were incubated with biotinylated anti-mouse IgG or anti-rabbit IgG (Vector Laboratories, Burlingame, Calif.) at 27° C. for 30 min and the antigen-antibody complex was detected with avidin-biotinylated horseradish peroxidase system (Vector, Burlingame, Calif., USA) using diaminobenzidine (DAB) as a chromogen and hematoxylin as a counterstain. Slides were evaluated as either negative or positive by an expert urologic pathologist.

Displayed were hematoxylin and eosin-stain and immunostaining for glutathione S-transferase-α (GST-α, F-H). A methylacyl racemase, carbonic anhydrase II (CAII), was demonstrated in normal renal cortex, clear cell RCC, papillary RCC and chromophobe RCC. Strong immunoreactivity was present in renal proximal and distal tubules, GST-α in clear cell RCC, AMACR in papillary RCC and CA H in chromophobe RCC.

### EXAMPLE III

### Classification of Kidney Tumors by Hierarchical Clustering

Hierarchical clustering (Eisen et al., supra) was used to classify kidney tumors based on their gene expression profiles using the expression ratios of a selected 3,560 cDNA set, as discussed in Example II. The clustering algorithm groups both genes and tumors by similarity in expression pattern. The patient dendrogram, which is based on expression profile of all 3,560 cDNAs is shown in FIG. 1. The gene expression pattern below the dendrogram was based on 1,309 genes that were statistically differentially expressed in each subtype compared to all other types of tumors. Two broad clusters emerged: one consisting of 35 clear cell RCC and 4 granular RCC, and the other all other types of kidney tumors plus 4 clear cell RCC. Five chromophobe RCC and 2 oncocytoma clustered together. The other clusters include 8 papillary RCC, 5 Wilms tumors, and 3 TCC. In the large cluster of clear cell RCC, there are two sub-clusters: one including all patients (except one) who died of cancer (E, FIG. 1) and the other the survivors of cancer without evidence of metastasis (D, FIG. 1). Two clear cell RCC, one primary tumor and a metastasized lymph node from the same patient were also examined (clear cell 40P, 40M). Interestingly, these two samples from the same patient had similar expression pattern, pointing to the genealogical relationship between the primary and metastatic tumor (Haddad 2002). A set of more highly expressed genes in each subtype of tumors compared to all other types of kidney tumors studied is indicated by side bars with different colors on the right-hand side of FIG. 1 (A: chromophobe RCC, B: papillary RCC, C: Wilms tumors, D: clear cell RCC with good outcome, E: clear cell RCC). Six granular cell RCC were located in a seemingly “random” fashion, suggesting it may not be a single entity. The diagnoses of these 6 cases were made in Japan prior to the recommendation of the work group of UICC and AJCC for RCC diagnosis. A blinded histological reevaluation was performed on 5 available cases by an expert urologic pathologist. “Granular RCC 1, 3 and 4”, which were clustered in clear cell RCC group, were re-classified as clear cell RCC. “Granular 2”, which was closely clustered with chromophobe RCC and oncocytomas, was re-classified as a chromophobe RCC. “Granular 5”, which has distinct histology, was not clustered with any RCC group by gene expression profile, may represent a novel subtype of RCC. These findings demonstrated the accuracy, objectivity and potential clinical utility of subclassifying kidney neoplasms by gene expression.

Multidimensional scaling (MDS) was then used to visualize the relationship among the profiles of all tumors. Three-dimensional (3D) visualization of the MDS data demonstrated how each RCC subtype clustered, e.g., chromophobe RCC/oncocytoma, papillary RCC, Wilms tumors, and TCC (FIG. 2A). “Granular 5”, which was of aggressive type and could not be re-classified, was placed next to the sarcomatoid RCC. Finally, the large majority of CC-RCC with poor outcome clustered to one side suggesting that they shared similar expression profiles (FIG. 2B).

### EXAMPLE IV

### Differentially Expressed Genes in Six Subtypes of Kidney Tumors

The global clustering analysis shown in Example III, using 3,560 cDNAs, showed that each of six subtypes of kidney tumors had distinct molecular signatures. In the present example, the differentially expressed genes contributing to these distinctions are identified.

CC RCC

Table 1 shows about 30 genes that are more highly expressed in clear cell RCC than in the other types of kidney tumors studied herein. The following are some overexpressed genes:

Peroxisome pioliferator-activated receptor gamma angiopoietin-related (PGAR), which was the most differentially expressed gene in CC-RCC (18.3 fold overexpression). Peroxisome proliferator-activated receptor-gamma (PPARγ) regulates adipose differentiation and systemic insulin signaling. PGAR has been found to be a target gene of PPARγ and the expression of PGAR is predominantly localized to adipose tissues and placenta. Also, it has been shown that hormone-dependent adipocyte differentiation occurs with early induction of the PGAR transcript (Yoon et al., Mol Cell Biol 2000; 20:5343-5349). The overexpression of this gene and the gene encoding adipose differentiation-related protein specific to clear cell RCC may be related to the abundance of cholesterol, cholesterol ester, and phospholipids in the cytoplasm of these cells. (Gonzalez et al., Invest Urol 1981; 19:1-3).

Vascular endothelial growth factor (VEGF) is shown to be highly expressed in CC-RCC and not in other RCC subtypes.

Glutathione S-transferase (GST)-α functions to protect the cell by catalyzing the detoxification of xenobiotics and carcinogens. Previous immunohistochemical studies have demonstrated strong expression in normal kidney, especially in the proximal tubules as well as in kidney cancer. We demonstrate here that its expression is specific in clear cell RCC and can be used as a marker in differentiating from other RCC subtypes. This is further confirmed by immunohistochemical staining (See, e.g., FIG. 3 and Table 4)

Five preferred genes whose increased expression is indicative of CC-RCC have been described above.

Papillary RCC

Table 2 shows about 30 genes that are more highly expressed in papillary RCC than in the other types of kidney tumors studied herein. Among the overexpressed genes are:

α-methylacyl coenzyme A racemase (AMACR). The enzyme encoded by the α-methylacyl coenzyme A racemase (AMACR) gene plays a critical role in peroxisomal P oxidation of branched chain fatty acid molecules. AMACR has been recently shown over-expressed in prostate cancer at both the transcript level by microarray experiments and the protein level (Rubin et al., JAMA 2002; 287(13):1662-70; Luo et al., Cancer Res 2002; 62(8):2220-6). Further studies by immunohistochemistry have demonstrated the elevation of AMACR protein in more than 90% of prostate cancer cases but not in benign prostatic tissues, suggesting that AMACR maybe a more specific marker than prostate specific antigen (PSA) for prostate cancer (Rubin, 2002, supra; Luo, 2002, supra). This gene was 5.3 times more highly expressed in papillary RCC. In addition, immunohistochemical analysis demonstrated immunoreactivity in 100% of papillary RCC cases, and less than 10% of other subtypes of RCC. (FIG. 3E-H).

TABLE 1
Relatively more highly expressed genes in clear cell RCC

| | NT SEQ | AA SEQ | | Fold | |
|---|---|---|---|---|---|
| Accession ID | ID NO: | ID NO: | Gene name | change | P Value |
| T54298 | 1 | 196 | PPAR (γ) angiopoietin related protein (PGAR) | 18.3 | 0.0001 |
| H95633 | 2 | 197 | crystallin, α A | 16.5 | 0.0001 |
| T73468 | 3 | 198 | glutathione S-transferase A2 | 11.4 | 0.0001 |
| N59772 | 4 | | ESTs- | 9.9 | 0.0001 |
| AA664406 | 5 | 199, 200 | complement component 4A | 9.7 | 0.0001 |
| AA668470 | 6 | 201 | regulator of G-protein signalling 5 | 8.8 | 0.0001 |
| AA169469 | 7 | 202 | pyruvate dehydrogenase kinase, isoenzyme 4 | 8.4 | 0.0001 |
| AA700054 | 8 | 203 | adipose differentiation-related protein | 8.0 | 0.0001 |
| H18608 | 9 | 204 | ESTs, Highly similar to organic anion transporter 3 | 7.9 | 0.0001 |
| AA150532 | 10 | 205 | keratin 6A | 7.6 | 0.0001 |
| H09076 | 11 | 206 | cytochrome P450, subfamily IIJ polypeptide 2 | 7.4 | 0.0001 |
| AA136707 | 12 | 207 | procollagen-lysine, 2-oxoglutarate 5-dioxygenase 2 | 7.2 | 0.0001 |
| W72294 | 13 | 208 | small inducible cytokine subfamily B, member 14 | 7.1 | 0.0001 |
| N30096 | 14 | 209 | glutathione S-transferase A3 | 6.6 | 0.0002 |
| AA454159 | 15 | 210 | H. sapiens HRBPiso mRNA, complete cds | 6.4 | 0.0001 |
| AA017544 | 16 | 211 | regulator of G-protein signalling 1 | 6.3 | 0.0001 |
| AA102107 | 17 | 212 | glutamyl aminopeptidase (aminopeptidase A) | 6.3 | 0.0001 |
| AA4880k70 | 18 | | immunoglobulin κ constant- | 6.2 | 0.0002 |
| N92646 | 19 | | colony stimulating factor 2 receptor, α, low-affinity- | 6.2 | 0.0001 |
| N93191 | 20 | | H. sapiens cDNA: FLJ22811 fis, clone KAIA2944 - | 6.1 | 0.0001 |
| R50354 | 21 | 213 | leukemia inhibitory factor (cholinergic differentiation | 5.9 | 0.0001 |
| | | | factor) | | |
| AA432292 | 2k2 | 214 | hypothetical protein DKFZp434F0318 | 5.8 | 0.0001 |
| T67053 | 23 | | immunoglobulin λ locus - | 5.7 | 0.0001 |
| AA486082 | 24 | 215 | serum/glucocorticoid regulated kinase | 5.6 | 0.0001 |
| AA598601 | 25 | | insulin-like growth factor binding protein 3 - | 5.6 | 0.0001 |
| N58170 | 26 | 216 | kidney- and liver-specific gene | 5.6 | 0.0002 |
| H15366 | 27 | | ESTs- | 5.3 | 0.0001 |
| H88329 | 28 | 217 | calbindin 1, (28 kD) | 5.2 | 0.0001 |
| H38650 | 29 | 218 | solute carrier family 2, member 5 | 5.1 | 0.0001 |
| R45059 | 30 | 219, 220 | vascular endothelial growth factor (VEGF) | 5.1 | 0.0001 |

The top 30 differentially expressed cDNAs in clear cell RCC are listed. They are significantly more highly expressed in clear cell RCC compared to all other types of kidney tumors studied by 10,000 times of permutation test. Fold change indicates clear cell RCC have relatively higher expression of this fold change compared to all other types of kidney tumors studied.

Guanine deaminase (GDA) is a DNA turnover enzyme and the gene encoding GDA was the most differentially expressed gene in papillary RCC. GDA activity has been found elevated in RCC (Durak et al., Cancer Invest 1997; 15(3):212-6) and gastric cancer (Durak et al., supra). GDA may be a useful marker for papillary RCC.

Another gene that is over-expressed in papillary RCC is Claudin-4, which is a member of a larger family of transmembrane tissue-specific claudin proteins that are essential components of intercellular tight junction structures. The gene is also over-expressed in prostate cancer (Long, et al., Cancer Res 2001; 61(21):7878-81) and pancreatic cancer (Michl et al., Gastroenterology 2001; 121(3):678-84). Two human dihydrodiol dehydrogenases, which are aldo-keto reductase family 1, member C1 (AKR1C1) and C3 (AK1RC3), were also highly expressed in papillary RCC. Both have been shown over-expressed in human prostate and mammary gland (Penning et al., Mol Cell Endocrinol 2001, 171: 137-149) and in non-small cell lung carcinoma (Hsu et al., Cancer Res 2001, 61:2727-2731) but have not been reported previously in papillary RCC.

Five preferred genes whose increased expression is indicative of papillary CC-RCC have been described above.

TABLE 2
Relatively more highly expressed genes in papillary RCC

| | NT SEQ | AA SEQ | | Fold | |
|---|---|---|---|---|---|
| Accession ID | ID NO: | ID NO: | GENE NAME | change | P Value |
| R60170 | 31 | 221 | Guanine deaminase | 18.0 | 0.0002 |
| W85851 | 32 | | H. sapiens Chromosome 16 BAC clone- | 10.6 | 0.0002 |
| H86812 | 33 | 222 | Heparan sulfate (glucosamine) 3-O-sulfotransferase 1 | 7.9 | 0.0001 |
| AA496334 | 34 | 223 | dynamin 1 | 7.7 | 0.0001 |
| AA873159 | 35 | 224 | apolipoprotein C-I | 6.8 | 0.0003 |
| AA459296 | 36 | 225 | solute carrier family 34, member 2 | 6.5 | 0.0001 |
| AA451904 | 37 | 226 | epididymis-specific, whey-acidic protein type | 6.4 | 0.00004 |
| R93124 | 38 | 227 | aldo-keto reductase family 1, member C1 | 5.7 | 0.0003 |
| AA135886 | 39 | 228 | H. sapiens mRNA; cDNA DKFZp434F053 | 5.5 | 0.0001 |
| AA127965 | 40 | | integrin, β 8 - | 5.3 | 0.0002 |
| AA453310 | 41 | 229 | α-methylacyl-CoA racemase | 5.2 | 0.0001 |
| AA916325 | 42 | 230 | aldo-keto reductase family 1, member C3 | 5.0 | 0.0004 |
| AA478724 | 43 | 231 | insulin-like growth factor binding protein 6 | 4.9 | 0.0001 |
| AA416585 | 44 | 232 | angiotensin I converting enzyme 2 | 4.8 | 0.0002 |
| R51836 | 45 | | H. sapiens clone CDABP0036 mRNA sequence - | 4.6 | 0.0002 |
| AA430665 | 46 | 233 | claudin 4 | 4.5 | 0.0002 |
| AA456022 | 47 | 234 | fibronectin leucine rich transmembrane protein 3 | 4.5 | 0.0003 |
| AA664101 | 48 | 235 | aldehyde dehydrogenase 1 family, member A1 | 3.9 | 0.0096 |
| R35051 | 49 | | ESTs- | 3.9 | 0.0001 |
| AA704995 | 50 | 236, 237, | putative glycine-N-acyltransferase | 3.8 | 0.0066 |
| | | 238 | | | |
| AA757672 | 51 | 239 | ESTs | 3.8 | 0.0001 |
| AA464688 | 52 | | ESTs, Weakly similar to unnamed protein product - | 3.7 | 0.0001 |
| AA292226 | 53 | 240 | accessory proteins BAP31/BAP29 | 3.6 | 0.0055 |
| AA437099 | 54 | | ESTs- | 3.6 | 0.0002 |
| AA406126 | 55 | 241 | Nit protein 2 | 3.5 | 0.0001 |
| AA489246 | 56 | 242 | suppression of tumorigenicity 14 | 3.5 | 0.0029 |
| H69786 | 57 | 243 | H. sapiens MAIL mRNA, complete cds | 3.5 | 0.0018 |
| T94781 | 58 | 244 | potassium inwardly-rectifying channel, subfamily J, | 3.5 | 0.0040 |
| | | | member 15 | | |
| AA455632 | 59 | 245 | chromosome 3p21.1 gene sequence | 3.4 | 0.0070 |
| AA644088 | 60 | 246, 247 | cathepsin C | 3.3 | 0.0006 |

The top 30 differentially expressed cDNAs in papillary RCC are listed. They are significantly more highly expressed in papillary RCC compared to all other types of kidney tumors studied by 10,000 times of permutation test. Fold change indicates papillary RCC have relatively higher expression of this fold change compared to all other types of kidney tumors studied.

Chromophobe RCC and Oncocytoma

Table 3 shows about 30 genes that are more highly expressed in chromophobe RCC and oncocytoma than in the other types of kidney tumors studied herein.

FIGS. 1 and 2 showed that five chromophobe RCC and two oncocytoma clustered close together, suggesting that these two subtypes have similar gene expression patterns. The similarity in expression profile between chromophobe RCC and oncocytoma has been previously reported (Young, 2001, supra).

It is known that chromophobe RCC/oncocytoma contain abundant mitochondria. Genes related to mitochondrial biology and oxidative phosphorylation were over-expressed in our study, suggesting the high specificity of these gene expression to chromophobe RCC/oncocytoma.

Carbonic anhydrases (CA) are a family of zinc metalloenzymes. CA IX has been shown to be tightly regulated by hypoxia-inducible factor-1 in renal carcinoma. CAII null mice have been shown to have renal tubular acidosis (Lewis et al., Proc Natl Acad Sci USA 1988; 85(6):1962-6) and the inability of acidifying urine (Brechue et al., Biochim Biophys Acta 1991; 1066(2):201-7). CAII have been shown expressed in tubular cells of the outer medulla and cortico-medullary junction by CAII gene delivery to CAII deficiency mice (Lai et al., J Clin Invest 1998; 101(7):1320-5). Our immunostaining confirmed the above findings in normal kidney and further demonstrated positivity in all chromophobe RCC (10/10) and oncocytomas (5/5). This marker is less specific than GST-α or AMACR because of its expression in small subsets of other renal tumors (Table 4).

Five preferred genes whose increased expression is indicative of chromophobe RCC/oncocytoma have been described above.

Table 5 shows genes that are more highly expressed in sarcomatoid than in the other types of kidney tumors studied herein.

We studied three mixed clear cell/sarcomatoid RCC and two sarcomatoid RCC. Among the differentially expressed genes is the SPARC (Secreted protein acidic and rich in cysteine) gene, whose sequence is found in GenBank as accession number AA436142 (SEQ ID NO:93). SPARC is associated with cell-matrix interactions during cell proliferation and extracellular remodeling. It is also implicated in the neovascularization, invasion, and metastasis of cancers the gene encoding SPARC was highly expressed in RCC with sarcomatoid component.

The genes encoding extracellular matrix compounds such as fibronectin (GenBank accession number R62612 (SEQ ID NO:92)) and collagen VI (GenBank accession number H99676 (SEQ ID NO:103)) were also found over-expressed in RCC with a sarcomatoid component in our study. Type VI collagen has been found widely distributed in RCC and fibronectin is an important stromal component especially in poorly differentiated carcinomas (Lohi et al., Histol Histopathlol 1998; 13(3):785-96). Another study has shown that the addition of the extracellular matrix compounds, fibronectin and collagen IV, resulted in a 5-10 fold increase in invasion of a RCC cell line. The over-expression of these genes in RCC with sarcomatoid component may underlie the behavior of sarcomatoid RCC, which has a high rate of metastasis and poor prognosis. These findings may elucidate the mechanisms of invasion and metastasis of sarcomatoid RCC.

Sarcomatoid RCC

Five preferred genes whose increased expression is indicative of chromophobe sarcomatoid RCC have been described above.

Other Type of Kidney Tumors

Transitional Cell Carcinoma (TCC)

Table 6 shows genes that are more highly expressed TCC than in the other types of kidney tumors studied herein.

TCC arising in the renal pelvis may invade throughout the entire kidney and as such, it may be difficult to distinguish TCC from RCC. Finding new markers for TCC may assist in its diagnosis. The gene encoding keratin 14 (GenBank accession number H44051 (SEQ ID NO:120)) is normally expressed in the basal cells of squamous epithelium. Keratin 14 has been proposed as a useful marker of squamous cell carcinoma (Chu et al., Histopathology 2001; 39(1):9-16). It has also been found expressed in TCC with squamous morphology and focally expressed in TCC with no morphological evidence of squamous differentiation (Harnden et al., J Clin Pathol 1997, 50:1032). Keratin 14, which was the most differentially expressed gene in our study, may serve as a useful marker for TCC of kidney. Several genes that were highly specific for TCC are related to skin. Collagen type VII (GenBank accession number AA598507 (SEQ ID NO:121)), for example, is the main constituent of anchoring fibrils, which are found below the basal lamina at the dermal-epidermal basement membrane zone in the skin (Sakai et al., j Cell Biol 1986; 103(4):1577-86). Keratin 19 (K19) (GenBank accession number AA464250 (SEQ ID NO:122) has been found in the periderm, the transient superficial layer that envelops the developing epidermis (Van Muijen et al., Exp Cell Res 1987; 171(2):331-45). By immunohistochemistry, we found K19 expression in some renal tubules, benign transitional epithelium and in 100% of 5 cases of TCC (Table 4 Integrin β-4 (GenBank accession number AA485668 (SEQ ID NO:125)) is expressed in human epidermis and restricted to the ventral surface opposed to the basal membrane zone. Integrin β-4 has been found to be associated with the hemidesmosomes in stratified and transitional epithelia (Jones et al., Cell Regul 1991; 2(6):427-38). Ladinin (GenBank accession number T97710 (SEQ ID NO:126)) is associated with the basement membrane located beneath hemidesmosomes (Moll et al., Virchows Arch 1998; 432(6):487-504). Taken together, these skin lesion-related genes may be specific markers for TCC of kidney.

Five preferred genes whose increased expression is indicative of TCC have been described above.

TABLE 3
Genes relatively more highly expressed in chromophobe RCC/oncocytoma

| | NT SEQ | AA SEQ | | Fold | |
|---|---|---|---|---|---|
| Accession ID | ID NO: | ID NO: | GENE NAME | change | P Value |
| H57180 | 61 | 248 | phospholipase C, γ 2 | 19.6 | 0.0001 |
| H23187 | 62 | 249 | carbonic anhydrase II | 13.8 | 0.0001 |
| AA399633 | 63 | | ESTs- | 9.9 | 0.0001 |
| N89673 | 64 | 250 | PPAR, γ, coactivator 1 | 9.2 | 0.0001 |
| W95082 | 65 | 251 | hydroxysteroid (11-β) dehydrogenase 2 | 9.0 | 0.0001 |
| N93505 | 66 | 252 | transmembrane 4 superfamily member 2 | 8.9 | 0.0001 |
| R59722 | 67 | | hypothetical protein FLJ10851 - | 8.3 | 0.0011 |
| T60160 | 68 | 253 | H. sapiens mRNA; cDNA | 7.6 | 0.0001 |
| H17036 | 69 | 254 | DHHC1 protein | 7.6 | 0.0001 |
| AA446650 | 70 | | H. sapiens mRNA; cDNA DKFZp586M0723 - | 7.5 | 0.0001 |
| R16134 | 71 | 255 | Plasmolipin | 7.2 | 0.0001 |
| AA406233 | 72 | 256 | ESTs, Highly similar to similar to GTPase-activating proteins | 7.1 | 0.0001 |
| T49816 | 73 | 257 | ESTs | 7.0 | 0.0001 |
| H22944 | 74 | 258 | nicotinamide nucleotide transhydrogenase | 6.9 | 0.0001 |
| R43873 | 75 | 259 | Human Chromosome 16 BAC clone CIT987SK-A-101F10 | 6.8 | 0.0001 |
| AA463445 | 76 | 260 | homolog of yeast ubiquitin-protein ligase Rsp5 | 6.7 | 0.0001 |
| N54401 | 77 | 261 | Rag D protein | 6.5 | 0.0001 |
| H22856 | 78 | 262 | glutamic-oxaloacetic transaminase 1, soluble | 6.3 | 0.0001 |
| R09053 | 79 | 263 | ESTs | 6.1 | 0.0001 |
| AA406362 | 80 | 264 | prostaglandin E receptor 3 (subtype EP3) | 6.1 | 0.0001 |
| H97921 | 81 | | ESTs - | 6.0 | 0.0001 |
| W31540 | 82 | | KIAA1450 protein - | 5.9 | 0.0001 |
| AA427619 | 83 | 265 | 1,2-α-mannosidase IC | 5.9 | 0.0001 |
| W47387 | 84 | | ecotropic viral integration site 5- | 5.7 | 0.0004 |
| N29800 | 85 | | hypothetical protein FLJ20783 - | 5.7 | 0.0001 |
| H99738 | 86 | 266 | Rag D protein | 5.7 | 0.0001 |
| AA894557 | 87 | 267 | Creatine kinase, brain | 5.7 | 0.0001 |
| AA452566 | 88 | 268 | Peroxisomal membrane protein 3 (35 kD) | 5.7 | 0.0001 |
| AA504265 | 89 | 260 | LIM and senescent cell antigen-like domains 1 | 5.6 | 0.0001 |
| AA682684 | 90 | 270 | Protein tyrosine phosphatase, non-receptor type 3 | 5.5 | 0.0001 |

The top 30 differentially expressed cDNAs in are listed. They are significantly more highly expressed in chromophobe RCC/oncocytoma compared to all other types of kidney tumors studied by 10,000 times of permutation test. Fold change indicates chromophobe RCC/oncocytoma have relatively higher expression of this fold change compared to all other types of kidney tumors studied.

TABLE 4
Immunohistochemical Reactivity of Four
Markers in 40 Primary Kidney Tumors

| | Clear | | Chromo- | Onco- | |
|---|---|---|---|---|---|
| | Cell | Papillary | phobe | cytoma | TCC |
| Marker | n = 10 | N = 10 | n = 10 | n = 5 | n = 5 |
| GST-α | 90% | 0% | 10% | 0% | ND |
| AMACR | 10% | 100% | 0% | 0% | ND |
| CA II | 30% | 10% | 100% | 100% | 20% |
| K19 | 0% | 10% | 0% | 0% | 100% |

TABLE 5
Relatively more highly expressed genes in sarcomatoid RCC

| | NT SEQ | AA SEQ | | # | Abs | ˜p | ˜FDR |
|---|---|---|---|---|---|---|---|
| UNIQID | ID NO | ID NO | GENE NAME | samples >1 | chg | value | (%) |
| AA670438 | 91 | | Ubiquitin carboxyl-terminal esterase L1 | 7 | 5.9 | 0.0009 | 0.8 |
| | | | (ubiquitin thiolesterase)- | | | | |
| R62612 | 92 | 271, 272 | Fibronectin 1 | 49 | 4.7 | 0.0081 | 2.3 |
| AA436142 | 93 | 273 | sparc/osteonectin, cwcv and kazal-like | 9 | 3.8 | 0.0021 | 1.1 |
| | | | domains proteoglycan (testican) | | | | |
| AA046525 | 94 | | H. sapiens, α-1 (VI) collagen- | 6 | 3.7 | 0.0019 | 1.1 |
| AA459305 | 95 | 274 | procollagen-lysine, 2-oxoglutarate 5- | 25 | 3.6 | 0.0001 | 0.3 |
| | | | dioxygenase 3 | | | | |
| AA487846 | 96 | | ESTs- | 36 | 3.5 | 0.0077 | 2.3 |
| AA464152 | 97 | 275 | quiescin Q6 | 15 | 3.4 | 0.0020 | 1.1 |
| W73810 | 98 | 276 | epithelial membrane protein 3 | 26 | 3.2 | 0.0008 | 0.8 |
| AA419177 | 99 | 277 | solute carrier family 7 (cationic amino | 17 | 2.9 | 0.0041 | 1.5 |
| | | | acid transporter, y+ system), member 5 | | | | |
| W45275 | 100 | 278 | CD44 antigen (homing function and | 21 | 2.9 | 0.0027 | 1.2 |
| | | | Indian blood group system) | | | | |
| AA678318 | 101 | 279 | hypothetical protein FLJ22341 | 12 | 2.7 | 0.0051 | 1.7 |
| H61003 | 102 | | EST- | 35 | 2.7 | 0.0078 | 2.2 |
| H99676 | 103 | 280 | collagen, type VI, α 1 | 13 | 2.7 | 0.0095 | 2.5 |
| AA448400 | 104 | 281 | plectin 1, intermediate filament binding | 17 | 2.6 | 0.0008 | 0.8 |
| | | | protein, 500 kD | | | | |
| AA504461 | 105 | 282 | low density lipoprotein receptor | 1 | 2.6 | 0.0006 | 0.8 |
| | | | (familial hypercholesterolemia) | | | | |
| AA521232 | 106 | 283 | HSPC022 protein | 14 | 2.5 | 0.0011 | 0.9 |
| AA402874 | 107 | 284 | phospholipid transfer protein | 12 | 2.3 | 0.0015 | 0.9 |
| AA426212 | 108 | 285 | Procollagen-proline, 2-oxoglutarate 4- | 33 | 2.3 | 0.0046 | 1.7 |
| | | | dioxygenase (proline 4-hydroxylase), β | | | | |
| | | | polypeptide (protein disulfide | | | | |
| | | | isomerase; thyroid hormone binding | | | | |
| | | | protein p55) | | | | |
| R44617 | 109 | 286 | MyoD family inhibitor | 14 | 2.3 | 0.0040 | 1.6 |
| W96107 | 110 | 287 | Sec61 γ | 20 | 2.3 | 0.0028 | 1.2 |
| AA186348 | 111 | 288, 289 | neuropathy target esterase | 5 | 2.2 | 0.0024 | 1.2 |
| H81907 | 112 | 290 | ankylosis, progressive (mouse) homolog | 4 | 2.2 | 0.0021 | 1.1 |
| N34466 | 113 | 291 | hypothetical protein DKFZp434 H0820 | 13 | 2.2 | 0.0019 | 1.1 |
| AA436406 | 1114 | 292 | N-myristoyltransferase 1 | 8 | 2.1 | 0.0025 | 1.2 |
| AA459400 | 115 | 293 | Rho GDP dissociation inhibitor (GDI) α | 8 | 2.1 | 0.0014 | 0.9 |
| AA454864 | 116 | 294 | ESTs, Weakly similar to A4P_human | 8 | 2 | 0.0013 | 0.9 |
| | | | intestinal membrane A4 protein | | | | |
| AA485714 | 117 | 295 | hypothetical protein FLJ22439 | 9 | 2 | 0.0093 | 2.5 |
| AA683550 | 118 | 296 | Interleukin-1 receptor-associated kinase 1 | 6 | 2 | 0.0018 | 1.1 |
| R17096 | 119 | | ESTs, Weakly similar to KE03 protein | 9 | 1.9 | 0.0034 | 1.4 |
| | | | [H. sapiens] | | | | |

TABLE 6
Relatively more highly expressed genes in TCC

| | SEQ | | # | abs | ˜p | ˜FDR |
|---|---|---|---|---|---|---|
| UNIQ ID | ID NO | NAME | samples >1 | chg | value | (%) |
| H44051 | 120 | keratin 14 (epidermolysis bullosa simplex, | 11 | 53.6 | 0.0001 | 0.3 |
| | | Dowling-Meara, Koebner) 17q12-q21 | | | | |
| AA598507 | 121 | collagen, type VII, α 1 (epidermolysis bullosa, | 11 | 18.3 | 0.0001 | 0.3 |
| | | dystrophic, dominant and recessive) | | | | |
| AA464250 | 122 | Keratin 19 | 15 | 14.4 | 0.0016 | 1 |
| N49853 | 123 | plexin B3 | 3 | 11.7 | 0.0004 | 0.5 |
| AA478481 | 124 | ESTs, Moderately similar to CA1C rat | 12 | 9.9 | 0.0016 | 1 |
| | | collagen α 1(XII) chain [R. norvegicus] | | | | |
| AA485668 | 125 | integrin, β 4 | 5 | 9.9 | 0.0001 | 0.3 |
| T97710 | 126 | ladinin 1 | 4 | 8.7 | 0.0001 | 0.3 |
| AA457728 | 127 | ESTs | 14 | 7.7 | 0.0005 | 0.5 |
| AA406020 | 128 | interferon-stimulated protein, 15 kDa | 22 | 5.8 | 0.0013 | 0.9 |
| AA457114 | 129 | tumor necrosis factor, α-induced protein 2 | 13 | 5.8 | 0.0011 | 0.8 |
| AA434390 | 130 | Hypothetical protein PRO0899 | 7 | 5.7 | 0.0027 | 1.2 |
| H22919 | 131 | cystatin B (stefin B) | 15 | 5.6 | 0.0002 | 0.4 |
| AA025408 | 132 | ESTs | 9 | 5.5 | 0.0006 | 0.6 |
| AA150053 | 133 | TEA domain family member 3 | 3 | 5.3 | 0.0001 | 0.3 |
| AA453783 | 134 | H. sapiens mRNA; cDNA DKFZp564B1264 | 2 | 4.9 | 0.0052 | 1.6 |
| | | (from clone DKFZp564B1264) | | | | |
| AA464731 | 135 | S100 calcium-binding protein A11 | 31 | 4.8 | 0.0023 | 1.1 |
| | | (calgizzarin) | | | | |
| N57743 | 136 | RelA-associated inhibitor | 9 | 4.8 | 0.0001 | 0.3 |
| AA426216 | 137 | malignant cell expression-enhanced | 5 | 4.5 | 0.0004 | 0.5 |
| | | gene/tumor progression-enhanced gene | | | | |
| H97778 | 138 | cadherin 1, type 1, E-cadherin (epithelial) | 8 | 4.5 | 0.0038 | 1.4 |
| AA430665 | 139 | claudin 4 | 10 | 3.9 | 0.0083 | 2.2 |
| AA022558 | 140 | H. sapiens cDNA: FLJ22120 fis, clone | 25 | 3.8 | 0.0003 | 0.4 |
| | | HEP 18874 | | | | |
| AA706987 | 141 | UDP-N-acetyl-α-D-galactosamine: polypeptide | 20 | 3.8 | 0.0002 | 0.4 |
| | | N-acetylgalactos_aminyltransferase 1 | | | | |
| | | (GalNAc-T1) | | | | |
| AA481745 | 142 | H. sapiens clone 23763 unknown mRNA, | 10 | 3.7 | 0.0002 | 0.4 |
| | | partial cds | | | | |
| R17096 | 143 | ESTs, Weakly similar to KE03 protein | 9 | 3.5 | 0.0006 | 0.6 |
| | | [H. sapiens] | | | | |
| H03961 | 144 | H. sapiens CAC-1 mRNA, partial cds | 15 | 3.3 | 0.0073 | 2 |
| AA436163 | 145 | prostaglandin E synthase | 4 | 3.2 | 0.0035 | 1.4 |
| AA455896 | 146 | glypican 1 | 14 | 3.2 | 0.0061 | 1.8 |
| AA406266 | 147 | Hypothetical protein FLJ23309 | 1 | 3.1 | 0.0037 | 1.4 |
| AA434159 | 148 | chromosome 19 open reading frame 3 | 5 | 3.1 | 0.0018 | 1 |
| H26294 | 149 | adaptor-related protein complex 1, γ2 subunit | 10 | 3.1 | 0.0002 | 0.4 |
| AA125872 | 150 | angiopoietin 2 | 13 | 3 | 0.0005 | 0.5 |
| AA436410 | 151 | branched chain aminotransferase 2, | 14 | 3 | 0.0028 | 1.2 |
| | | mitochondrial | | | | |
| AA485734 | 152 | Ran GTPase activating protein 1 | 4 | 3 | 0.0002 | 0.4 |
| AA620747 | 153 | ESTs | 4 | 3 | 0.0039 | 1.4 |
| H15456 | 154 | calpain 1, (mu/I) large subunit | 8 | 3 | 0.0018 | 1 |
| W95682 | 155 | H. sapiens cDNA FLJ20153 fis, clone | 28 | 3 | 0.0009 | 0.7 |
| | | COL08656, highly similar to AJ001381 H. | | | | |
| | | sapiens incomplete cDNA for a mutated allele | | | | |
| AA001718 | 156 | ESTs | 5 | 2.9 | 0.0020 | 1 |
| AA455284 | 157 | hypothetical protein | 4 | 2.9 | 0.0001 | 0.3 |
| H18080 | 158 | H. sapiens mRNA; cDNA DKFZp667O2416 | 4 | 2.9 | 0.0011 | 0.8 |
| | | (from clone DKFZp667O2416) | | | | |
| H44956 | 159 | fumarylacetoacetate | 4 | 2.9 | 0.0042 | 1.4 |
| AA598513 | 160 | protein tyrosine phosphatase, receptor type, F | 11 | 2.8 | 0.0006 | 0.6 |
| H99033 | 161 | EST | 5 | 2.8 | 0.0004 | 0.5 |
| AA047443 | 162 | LIM domain-containing preferred translocation | 2 | 2.7 | 0.0028 | 1.2 |
| | | partner in lipoma | | | | |
| AA459381 | 163 | AA459381 sphingosine-1-phosphate lyase 1 | 3 | 2.7 | 0.0015 | 0.9 |
| AA707696 | 164 | COBW-like protein | 2 | 2.6 | 0.0002 | 0.4 |
| AA877255 | 165 | interferon regulatory factor 7 | 3 | 2.6 | 0.0063 | 1.8 |
| N45236 | 166 | N45236 ESTs | 2 | 2.6 | 0.0020 | 1 |
| AA131707 | 167 | ESTs | 3 | 2.5 | 0.0007 | 0.6 |
| AA464963 | 168 | ESTs | 4 | 2.5 | 0.0040 | 1.4 |
| AA878576 | 169 | chromosome 19 open reading frame 3 | 8 | 2.5 | 0.0001 | 0.3 |
| H56069 | 170 | H56069 glutamate-cysteine ligase, catalytic | 1 | 2.5 | 0.0011 | 0.8 |
| | | subunit | | | | |
| H65395 | 171 | proteasome (prosome, macropain) activator | 10 | 2.5 | 0.0012 | 0.8 |
| | | subunit 2 (PA28 β) | | | | |
| AA046043 | 172 | endosulfine α | 2 | 2.4 | 0.0013 | 0.9 |
| AA401972 | 173 | RAB2, member RAS oncogene family-like | 1 | 2.4 | 0.0045 | 1.4 |
| AA430576 | 174 | KIAA0657 protein | 2 | 2.4 | 0.0088 | 2.3 |
| AA496541 | 175 | KIAA0317 gene product | 0 | 2.4 | 0.0080 | 2.1 |
| AA459658 | 176 | ESTs | 2 | 2.3 | 0.0007 | 0.6 |
| AA669042 | 177 | actinin, α 1 | 9 | 2.3 | 0.0080 | 2.1 |
| AA706829 | 178 | utative Rab5-interacting protein | 11 | 2.3 | 0.0056 | 1.6 |
| H29625 | 179 | hypothetical protein FLJ20411 | 5 | 2.3 | 0.0022 | 1.1 |
| AA156793 | 180 | AA156793 nuclear receptor coactivator 3 | 6 | 2.2 | 0.0044 | 1.4 |
| AA679352 | 181 | farnesyl-diphosphate farnesyltransferase 1 | 3 | 2.2 | 0.0015 | 0.9 |
| H42874 | 182 | ubiquitin specific protease 21 | 2 | 2.2 | 0.0051 | 1.6 |
| H56903 | 183 | H. sapiens mRNA; cDNA DKFZp434A1114 | 7 | 2.2 | 0.0077 | 2.1 |
| | | (from clone DKFZp434A1114) | | | | |
| N50834 | 184 | mevalonate (diphospho) decarboxylase | 3 | 2.2 | 0.0039 | 1.4 |
| AA427887 | 185 | KIAA1436 protein | 21 | 2.1 | 0.0044 | 1.4 |
| AA453512 | 186 | diacylglycerol O-acyltransferase (mouse) | 7 | 2.1 | 0.0018 | 1 |
| | | homolog | | | | |
| AA454556 | 187 | hypothetical protein FLJ10767 | 9 | 2.1 | 0.0030 | 1.3 |
| R74078 | 188 | H. sapiens mRNA for KIAA1741 protein, | 8 | 2.1 | 0.0019 | 1 |
| | | partial cds | | | | |
| W89187 | 189 | brefeldin A-inhibited guanine nucleotide- | 2 | 2.1 | 0.0053 | 1.6 |
| | | exchange protein 1 | | | | |
| AA459399 | 190 | AA459399 KIAA0356 gene product | 2 | 2 | 0.0069 | 1.9 |
| AA459402 | 191 | KIAA1631 protein | 5 | 2 | 0.0040 | 1.4 |
| H19340 | 192 | H19340 membrane interacting protein of | 8 | 2 | 0.0096 | 2.4 |
| | | RGS16 | | | | |
| AA191356 | 193 | eukaryotic translation initiation factor 4 γ, 2 | 2 | 1.9 | 0.0097 | 2.4 |

Wilms' Tumors (WT)

Insulin-like growth factor II (IGF II) gene (GenBank accession number N74623 (SEQ ID NO:195)) is one of the differentially expressed genes in WT. IGF II is located on chromosome 11p15, which is usually imprinted (only expressed in the paternally derived allele). In Beckwith-Wiedeman disease, a hereditary form of WT, some patients constitutionally lose the imprinting of IGF II. Some sporadic WT also show the loss of imprinting of IGF II and this may result in high expression of IGF H in WT.

Glypican 3 (GenBank accession number AA775872 (SEQ D NO: 194)) is a heparan sulfate proteoglycan and usually expressed in the fetal mesodermal tissue. Its disruption leads to gigantism or overgrowth. In this study, glypican 3 was the most differentially expressed gene in WT High expression of IGFII and glypican 3 may be a specific characteristic in WT.

From the foregoing description, one skilled in the art can easily ascertain the essential characteristics of this invention, and without departing from the spirit and scope thereof, can make changes and modifications of the invention to adapt it to various usage and conditions.

Without further elaboration, one skilled in the art can, using the preceding description, utilize the present invention to its fullest extent. The preferred specific embodiments disclosed above are to be construed as merely illustrative, and are not intended to limit the scope of the invention.

The entire disclosure of all patent applications, patents and other publications, cited above and in the figures are hereby incorporated by reference in their entirety.

1-28. (canceled)

29. A composition comprising:
(a) one, two, three, four or five isolated nucleic acids represented by SEQ ID NO:1; SEQ ID NO:2; SEQ ID NO:3; SEQ ID NO:5; and/or SEQ ID NO:6; or fragments thereof that comprise at least about 10 contiguous nucleotides of said sequences, and/or
(b) one, two, three, four or five isolated nucleic acids represented by SEQ ID NO:31; SEQ ID NO:33; SEQ ID NO:34; SEQ ID NO:35; and/or SEQ ID NO:36; or fragments thereof that comprise at least about 10 contiguous nucleotides of said sequences, and/or
(c) one, two, three, four or five isolated nucleic acids represented by SEQ ID NO:61; SEQ ID NO:62; SEQ ID NO:64; SEQ ID NO:65; and/or SEQ ID NO:66; or fragments thereof that comprise at least about 10 contiguous nucleotides of said sequences, and/or
(d) one, two, three, four or five isolated nucleic acids represented by SEQ ID NO:91; SEQ ID NO:92; SEQ ID NO:93; SEQ ID NO:94; and/or SEQ ID NO:95; or fragments thereof that comprise at least about 10 contiguous nucleotides of said sequences, and/or
(e) one, two, three, four or five isolated nucleic acids represented by SEQ ID NO:120; SEQ ID NO:121; SEQ ID NO:122; SEQ ID NO:123; and/or SEQ ID NO:125; or fragments thereof that comprise at least about 10 contiguous nucleotides of said sequences, and/or
(f) one or two isolated nucleic acids represented by SEQ ID NO:194 and/or SEQ ID NO:195, or fragments thereof that comprise at least about 10 contiguous nucleotides of said sequences.

30. The composition of claim 29, wherein each of (a), (b), (c), (d) and (e) comprises all five of the indicated nucleic acids and (f) comprises both of said nucleic acids.

31. The composition of claim 1, which is in the form of an aqueous solution.

32. The composition of claim 1, which is in the form of an array.

33. The array of claim 32, which comprises at least about 900 nucleic acids.

34. A composition comprising a set of two or more nucleic acid probes, each of which hybridizes with part or all of a coding sequence that is overexpressed in clear cell renal cell carcinoma (CC-RCC), papillary RCC, chromophobe/oncocytoma RCC, sarcomatoid RCC, TCC, or Wilms' tumors, which overexpression is based on comparison to a baseline value.

35. The composition of claim 34, wherein the baseline value is the expression of said coding sequence in normal renal tissue from (i) the subject from whom the tumor tissue is obtained or (ii) one or more normal individuals.

36. The composition of claim 34, which is in the form of an array.

37. The composition of claim 35, which is in the form of an array.

38. The composition of claim 29, wherein one or more of the nucleic acids comprise nucleotides having at least one modified phosphate backbone selected from a phosphorothioate, a phosphoridothioate, a phosphoramidothioate, a phosphoramidate, a phosphordiimidate, a methylsphosphonate, an alkyl phosphotriester, 3′-aminopropyl, a formacetal, or an analogue thereof.

39. The composition of claim 34, wherein one or more of the nucleic acids comprise nucleotides having at least one modified phosphate backbone selected from a phosphorothioate, a phosphoridothioate, a phosphoramidothioate, a phosphoramidate, a phosphordiimidate, a methylsphosphonate, an alkyl phosphotriester, 3′-aminopropyl, a formacetal, or an analogue thereof.

40. The array of claim 32 further comprising, bound to one or more nucleic acids of the array, one or more polynucleotides from a sample representing expressed genes, wherein the sample is from an individual subject's renal tumor, from normal tissue, or from both tumor and normal tissue.

41. The array of claim 36, further comprising, bound to one or more nucleic acids of the array, one or more polynucleotides from a sample representing expressed genes, wherein the sample is from an individual subject's renal tumor, from normal tissue, or from both tumor and normal tissue.

42. The array of claim 32, wherein the nucleic acids of the array have been hybridized under conditions of high stringency to one or more polynucleotides from a sample representing expressed genes, wherein the sample is from an individual subject's renal tumor, from normal tissue, or from both tumor and normal tissue.

43. The array of claim 36, wherein the nucleic acids of the array have been hybridized under conditions of high stringency to one or more polynucleotides from a sample representing expressed genes, wherein the sample is from an individual subject's renal tumor, from normal tissue, or from both tumor and normal tissue.

44. The composition of claim 29, wherein the isolated nucleic acids are of human origin.

45. The composition of claim 34, wherein the isolated nucleic acids are of human origin.

46. A composition comprising
(a) one, two, three, four or five of the following isolated polypeptides: SEQ ID NO:196; SEQ ID NO:197; SEQ ID NO:198; SEQ ID NO:199 or 200; and/or SEQ ID NO:201, or an antigenic fragment[s] of said polypeptide, and/or
(b) one, two, three, four or five of the following isolated polypeptides: SEQ ID NO:221; SEQ ID NO:222; SEQ ID NO:223; SEQ ID NO:224; and/or SEQ ID NO:225, or an antigenic fragment[s] of said polypeptide, and/or
(c) one, two, three, four or five of the following isolated polypeptides: SEQ ID NO:248; SEQ ID NO:249; SEQ ID NO:250; SEQ ID NO:251; and/or SEQ ED NO:252, or an antigenic fragment[s] of said polypeptide, and/or
(d) one, two, three, four or five of the following isolated polypeptides: (i) a polypeptide encoded by an open reading frame (ORF) that includes the nucleotide sequence SEQ ID NO:91; (ii) SEQ ID NO:271 or 272; (iii) SEQ ID NO:273; (iv) a polypeptide encoded by an ORF of SEQ ID NO:94; and/or (v) SEQ ID NO:274, or antigenic fragments thereof, and/or
(e) one, two, three, four or five polypeptides encoded by the following nucleic acids: (i) an ORF that includes SEQ ID NO:120; (ii) SEQ ID NO:121; (iii) SEQ ID NO:122; (iv) SEQ ID NO:123; and (v) SEQ ID NO:125; or an antigenic fragment[s] of said polypeptide, and/or
(f) one or two isolated polypeptides encoded by the nucleic acids SEQ ID NO:194 and/or SEQ ID NO:195; or an antigenic fragment[s] of said isolated polypeptide.

47. The composition of claim 46, wherein each of (a), (b), (c), (d) and (e) comprises all five of the indicated polypeptides or antigenic fragments, and (f) comprises both of said polypeptides or antigenic fragments.

48. A composition comprising antibodies specific for the polypeptides or fragments of the composition of claim 46.

49. The composition of claim 46, which is in the form of an array.

50. The composition of claim 47, which is in the form of an array.

51. The composition of claim 48, which is in the form of an array.

52. A method for determining the subtype of a renal carcinoma in a subject, comprising
(a) hybridizing nucleic acids of the composition of claim 29, under conditions of high stringency, to polynucleotides of a sample of the renal carcinoma; and
(b) comparing the amount of the sample polynucleotides hybridized to said nucleic acids of the composition, to a baseline value,
wherein the amount of sample polynucleotide hybridized is indicative of the level of expression of the polynucleotide or polynucleotides in the renal tumor, and wherein said level of expression is characteristic of the subtype of renal carcinoma.

53. The method of claim 52, wherein the nucleic acid composition is in the form of an array.

54. The method claim 52, wherein,
(a) when the expression of said sample polynucleotide, as determined by its hybridization to one or more nucleic acids listed in Table 1, is up-regulated compared to the baseline value, the renal tumor is a clear cell-RCC;
(b) when the expression of said sample polynucleotide, as determined by its hybridization to one or more nucleic acids listed in Table 2, is up-regulated compared to the baseline value, the renal tumor is a papillary RCC;
(c) when the expression of said sample polynucleotide, as determined by its hybridization to one or more nucleic acids listed in Table 3, is up-regulated compared to the baseline value, the renal tumor is chromophobe-RCC/oncocytoma;
(d) when the expression of said sample polynucleotide, as determined by its hybridization to one or more nucleic acids listed in Table 5, is up-regulated compared to the baseline value, the renal tumor is a sarcomatoid-RCC;
(e) when the expression of said sample polynucleotide, as determined by its hybridization to one or more nucleic acids listed in Table 6, is up-regulated compared to the baseline value, the renal tumor is a transitional cell carcinoma; and
(f) when the expression of said sample polynucleotide, as reflected by its hybridization to one or more nucleic acids represented by SEQ ID NO:194 or SEQ ID NO:195, is up-regulated compared to the baseline value, the renal tumor is a Wilms' tumor.

55. The method claim 53, wherein,
(a) when the expression of said sample polynucleotide, as determined by its hybridization to one or more nucleic acids listed in Table 1, is up-regulated compared to the baseline value, the renal tumor is a clear cell-RCC;
(b) when the expression of said sample polynucleotide, as determined by its hybridization to one or more nucleic acids listed in Table 2, is up-regulated compared to the baseline value, the renal tumor is a papillary RCC;
(c) when the expression of said sample polynucleotide, as determined by its hybridization to one or more nucleic acids listed in Table 3, is up-regulated compared to the baseline value, the renal tumor is chromophobe-RCC/oncocytoma;
(d) when the expression of said sample polynucleotide, as determined by its hybridization to one or more nucleic acids listed in Table 5, is up-regulated compared to the baseline value, the renal tumor is a sarcomatoid-RCC;
(e) when the expression of said sample polynucleotide, as determined by its hybridization to one or more nucleic acids listed in Table 6, is up-regulated compared to the baseline value, the renal tumor is a transitional cell carcinoma; and
(f) when the expression of said sample polynucleotide, as reflected by its hybridization to one or more nucleic acids represented by SEQ ID NO:194 or SEQ ID NO:195, is up-regulated compared to the baseline value, the renal tumor is a Wilms' tumor.

56. The method of claim 52, wherein said sample polynucleotide is labeled with a detectable label.

57. The method of claim 56, wherein the detectable label is a fluorescent label.

58. A method for determining the subtype of a renal carcinoma in a subject, comprising
(a) contacting the antibody composition of claim 48 with a polypeptide sample obtained from the renal carcinoma, under conditions effective for an antibody to bind specifically to a polypeptide; and
(b) comparing the amount of said binding to a baseline value,
wherein the amount of binding of said sample polypeptide to said specific antibody or antibodies of said composition is indicative of the level of expression of the polypeptide in the renal tumor, and wherein said level of expression is characteristic of the subtype of renal carcinoma.

59. A kit for detecting the presence and/or amount of a polynucleotide in a renal tumor sample, which presence and or/amount is indicative of a subtype of renal carcinomas, the kit comprising:
(a) the nucleic acid composition of claim 29; and, optionally,
(b) one or more reagents that facilitates hybridization of nucleic acids of the composition to the sample polynucleotide, and/or that facilitates detection of the hybridized polynucleotide.

60. A kit for detecting the presence and/or amount of a polynucleotide in a renal tumor sample, which presence and or/amount is indicative of a subtype of renal carcinomas, the kit comprising:
(a) the nucleic acid composition of claim 34; and, optionally,
(b) one or more reagents that facilitates hybridization of nucleic acids of the composition to the sample polynucleotide, and/or that facilitates detection of the hybridized polynucleotide.

61. The kit of claim 59, wherein the nucleic acid composition is in the form of an array of said nucleic acids.

62. The kit of claim 60, wherein the nucleic acid composition is in the form of an array of said nucleic acids.

63. A kit for detecting the presence and/or amount of a polypeptide in a renal tumor sample, which presence and/or amount is indicative of subtype of renal carcinoma, comprising:
(a) the antibody composition of claim 48; and, optionally,
(b) one or more reagents that facilitates binding of the antibodies of the composition to the sample polypeptide, and/or that facilitates detection of antibody binding.

64. The kit of claim 63, wherein the antibody composition is in the form of an array of said antibodies.
